(19) United States
(12) Patent Application Publication
(10) Pub. No.: US 2005/0085426 A1
(43) Pub. Date: Apr. 21, 2005

(54) Acid-sensitive compounds, preparation and use thereof

(76) Inventors: Michel Bessodes, Villejuif (FR); Christophe Masson, Montgeron (FR); Daniel Scherman, Paris (FR); Barbara Wetzer, Vienna (AT)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/129,262

(22) PCT Filed: Sep. 3, 2001
(86) PCT No.: PCT/FR01/02725

Related U.S. Application Data
(60) Provisional application No. 60/239,116, filed on Oct. 11, 2000.

(30) Foreign Application Priority Data
Sep. 5, 2000 (FR) 0011278

(51) Int. Cl. A61K 31/70

(52) U.S. Cl. 514/25

(57) ABSTRACT

Novel acid-sensitive compounds comprising at least one hydrophilic substituent and a cyclic ortho-ester which is acid-sensitive, and their salts. These compounds are useful for forming conjugates (liposomes, complexes, nanoparticles and the like) with biologically active substances and releasing them into cellular tissues or compartments whose pH is acidic, or as nonionic surfactant for stabilizing particles encapsulating a biologically active substance and then destabilizing them in acid medium, or alternatively as a vector covalently linked to a therapeutic molecule so as to release said therapeutic molecule into the cellular tissues or compartments whose pH is acidic.

The present invention relates to acid-sensitive compounds and their preparation. These compounds comprise at least one hydrophilic substituent and a cyclic ortho-ester which is acid-sensitive. These compounds are useful for forming conjugates (liposomes, complexes, nanoparticles and the like) with biologically active substances and releasing them into cellular tissues or compartments whose pH is acidic, or as nonionic surfactant for stabilizing particles encapsulating a biologically active substance and then destabilizing them in acid medium, or alternatively as a vector covalently linked to a therapeutic molecule so as to release it into the cellular tissues or compartments whose pH is acidic.

The release of biologically active substances into tissues or cells having an increased acidity relative to what is physiologically normal is a known problem which has been the subject of numerous studies, without as a result giving completely satisfactory results up until now. Thus, many pH-sensitive liposomes have been designed so as to release biologically active substances by taking advantage of the acidification of certain tissues or of the endosome.

For example, Yatvin et al. (Science, Vol. 210, 1980, pp. 1253-4) designed pH-sensitive lipids capable of becoming inserted into the lipid bilayer of conventional liposomes, of formula:

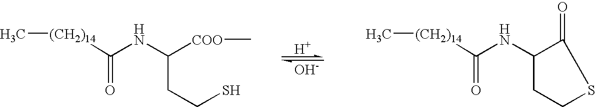

The homocysteine present in this lipid is in its open form at neutral or alkaline pH and resembles, in this case, a fatty acid which becomes perfectly inserted into the bilayer of the liposomes. At this pH, it exists in its closed form: it then forms a cyclic thiolactone, thus resembling a neutral lipid which destabilizes the liposomal bilayer and thus allows the release of the active substance. Such a molecule allows the release of medicinal molecules in the regions of the body in which the pH is less than the physiological pH, for example in primary tumors, metastases, or alternatively sites of inflammation and of infection.

American patent U.S. Pat. No. 5,965,434 proposes amphiphatic lipids comprising a cationic pH-sensitive hydrophilic part of formula: 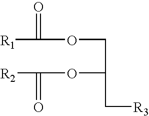
in which R₁ and R₂ represent, independently of each other, CH₃(CH₂)₁₄, CH₃(CH₂)₁₂, CH₃(CH₂)₇CHCH(CH₂)₇, and R₃ represents a substituent 1-methylimidazole, imidazole, 4,9-dioxo-1,12-dodecanediamine, cysteamine, 1-(3-aminopropyl)imidazole, morpholine, 4-aminopyridine, pyridine, guanidine, hydrazine, thiouronium or piperazine.

These compounds have the characteristic feature of carrying an overall positive charge (at the level of the compound R₃) which increases when the pH decreases from 8.0 to 4.5. This modification of the charge induces a conformational transformation of the liposome, allowing it to release its content. These lipids thus allow the release of medicinal molecules or of nucleic acids into acidic media whose pH varies up to 4.5.

Moreover, application WO 97/31624 proposes pH-sensitive phospholipids (“triggerable lipids”) which comprise a vinyl ether function which may be degraded in the cytoplasm, and which have the general formula: 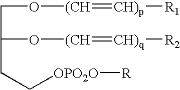
in which p and q are equal to 0 or 1, at least one of the two being equal to 1, R₁ and R₂ represent, independently of each other, an alkyl or an alkene containing 12 to 24 carbon atoms, and R represents a group chosen from 2-aminoethyl, 2-(trimethylamino)-ethyl, 2-(N,N-dimethylamino)ethyl, 2-(trimethyl-ammonium)ethyl, 2-carboxy-2-aminoethyl, succinamido-ethyl or inosityl.

These phospholipids are mixed with other phospholipids, which are themselves complexed with cell receptor ligands, so as to form liposomes capable of undergoing conformational changes at acidic pH. Such liposomes allow the encapsulation of numerous medicinal substances and also of nucleic acids for gene therapy.

Another approach has also been described (Kratz et al., Crit. Rev. Ther. Drug Carrier Syst. 1999, 16(3), pp. 245-88) in which a therapeutic molecule is covalently linked to a polymer via an acid-sensitive bond so as to ensure the release of said therapeutic molecule into weakly acidic tumor tissues or alternatively into the endosomes and lysosomes after cellular internalization of the polymeric conjugate. Numerous possible bonds have thus been described, for example acetal, disulfide, hydrazone, cis-aconitrile, trityl or alternatively silylated ether bonds.

However, all the pH-sensitive compounds developed up until now have the disadvantage of not being modulable as regards their sensitivity. Thus, it would be highly advantageous to be able to have acid-labile compounds whose sensitivity could be modulated according to, for example, the tissues or cells targeted, the biologically active substance to be released or alternatively the applications envisaged. More generally, it would also be advantageous to be able to have new pH-sensitive compounds which would be easy to prepare and effective for the transfection of nucleic acids in particular.

To solve this problem, the Applicant has thus developed a novel family of acid-sensitive compounds characterized in that they comprise a cyclic ortho-ester and at least one hydrophilic substituent chosen from polyalkylene glycols, mono- or polysaccharides, hydrophilic therapeutic molecules, or radicals of the polyamine type.

Such compounds are useful for the vectorization and the release of biologically active substances into the acidic regions of the body by virtue of the cyclic ortho-ester function which is acid-sensitive. They are most particularly advantageous because the pH-sensitivity of the compound may be modulated according to the choice of the substituent present on the central carbon and the size of the ortho-ester ring. It is thus possible to broadly vary the kinetics of hydrolysis of these compounds and therefore to modulate the time necessary for the release of the biologically active substance. In addition, the acid-sensitive compounds according to the present invention have the additional advantage of becoming degraded in acidic medium in an autocatalytic manner. Indeed, the partial degradation of the acid-sensitive compounds according to the invention causes the gradual release of an acid (for example formic acid when the starting compound is derived from an ortho-formate, or alternatively acetic acid when the starting compound is derived from an ortho-acetate, or alternatively benzoic acid when the starting compound is derived from an ortho-benzoate) which induces a decrease in the pH, further promoting their degradation.

More particularly, the acid-sensitive compounds according to the present invention have the general formula: 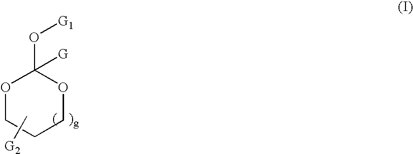
in which:
g is an integer which may take the values 0, 1, 2, 3 or 4,
G represents a hydrogen atom, an alkyl radical containing 1 to 6 carbon atoms in the form of a saturated or unsaturated, a straight or branched chain, or an aryl radical,
G₁ and G₂represent:
(a) one a hydrophilic substituent chosen from radicals of the polyamine type, and the other a hydrophobic substituent chosen from single- or double-chain alkyls, steroid derivatives or hydrophobic dendrimers, or alternatively
(b) one a hydrophobic linear alkyl group comprising 10 to 24 carbon atoms and optionally comprising one or more unsaturations, and the other a group of general formula: 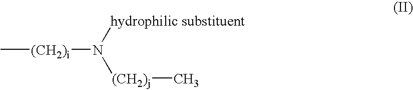
   in which i is an integer chosen from between 1 and 4 inclusive and j is an integer chosen from between 9 and 23 inclusive, and the hydrophilic substituent is chosen from radicals of the polyamine type, or alternatively
(c) one a hydrophilic substituent chosen from polyalkylene glycols or mono- or polysaccharides and the other a substituent chosen from polyalkylene imines, or alternatively
(d) one a hydrophilic substituent chosen from polyalkylene glycols or mono- or polysaccharides and the other a hydrophobic substituent chosen from single- or double-chain alkyls, steroid derivatives, hydrophobic dendrimers, or the covalent conjugates between a single- or double-chain alkyl, a steroid derivative, or a hydrophobic dendrimer and a polyalkylene glycol molecule comprising 1 to 20 monomeric units, or alternatively
(e) one a hydrophilic substituent chosen from polyalkylene glycols or mono- or polysaccharides and the other a therapeutic molecule, or alternatively
(f) one a therapeutic molecule of a hydrophilic nature and the other a hydrophobic substituent chosen from single- or double-chain alkyls, steroid derivatives or hydrophobic dendrimers.

The substituent G placed on the central carbon of the ortho-ester is chosen so as to modulate the sensitivity of the acid-sensitive compound according to the present invention. Thus, the more electron-donating the group G, the more acid-sensitive the compound, and the more electron-attracting the group G, the less acid-sensitive the compound. It seems, therefore, that the choice of the radical G is particularly important for determining and modulating the properties of the acid-sensitive compounds of general formula (I). According to a preferred aspect of the invention, G is chosen from the hydrogen atom, the alkyl radicals comprising 1 to 6 carbon atoms in the form of a saturated or unsaturated, straight or branched chain, or aryl radicals. In a more particularly advantageous manner, G is chosen from hydrogen, methyl, ethyl or phenyl.

For the purposes of the present invention, the expression “aryl radicals” is understood to mean univalent aromatic hydrocarbon radicals. The aryl radicals according to the present invention generally contain between 6 and 14 carbon atoms. Preferably, the aryl radicals according to the present invention are chosen from phenyl, naphthyl, for example 1-naphthyl or 2-naphthyl, biphenylyl, for example 2-biphenylyl, 3-biphenylyl or 4-biphenylyl, anthryl or fluorenyl. The phenyl is more particularly preferred. The aryl radicals, in particular the phenyl, may be substituted or otherwise, for example monosubstituted, disubstituted, trisubstituted or tetrasubstituted, the substituents being identical or different. Preferably, said substituents are chosen from halogen atoms, (C₁-C₈)alkyl or (C₁-C₈)alkoxy radicals. In the case of monosubstituted phenyl radicals, said substituent may be substituted at position 2, at position 3 or at position 4. In the case of disubstited phenyl radicals, said substituents may be situated at position 2,3, at position 2,4, at position 2,5, at position 2,6, at position 3,4 or at position 3,5. In the case of trisubstituted phenyl radicals, said substituents may be situated, for example, at position 2,3,4, at position 2,3,5, at position 2,4,5, at position 2,4,6, at position 2,3,6 or at position 3,4,5.

Depending on the cases, each of the substituents G₁ and G₂ is either directly linked to the cyclic ortho-ester, or indirectly via a “spacer” molecule chosen from those known to persons skilled in the art. Such a “spacer” molecule makes it possible both to ensure the binding and to move the substituent(s) in question away from the cyclic ortho-ester in order to reduce any undesirable interaction between the acid-sensitive cyclic ortho-ester and its subsituent(s). Preferred spacer molecules may be chosen for example according to the nature of the substituents G₁ or G₂ from alkyls (1 to 6 carbon atoms), carbonyl, ester, ether, amide, carbamate or thiocarbamate bonds, glycerol, urea, thiourea or a combination of several of these groups. For example, when the hydrophobic substituent is a steroid derivative, the spacer molecule may be a bond of the carbamate —N—C(O)—O— type, or alternatively when the hydrophobic substituent is a double-chain alkyl, the spacer molecule may be chosen from the groups of formula -alkyl-C(O)—N, the two alkyl chains then being fixed to the nitrogen atom.

According to a preferred aspect of the invention, the radicals of the polyamine type may be defined as being linear or branched alkyls comprise at least 3 carbon atoms and in which at least one of the methylene groups may be replaced with an amino group which is optionally substituted (with a methyl group for example) and the terminal methyl(s) is(are) substituted with one or more groups chosen from (primary, secondary, tertiary or quaternary) amines, guanidines or cyclic guanidines. These radicals of the polyamine type are preferably chosen from the polyamine radicals which are already known and described in the literature for the vectorization of nucleic acids, for example in the publications WO 96/17823, WO 97/18185, WO 98/54130 or alternatively WO 99/51581. In particular, this may include for example polyamines of general formula: 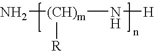
in which
R represents a hydrogen atom with the exception of only one of the groups R which is absent and therefore represents the covalent bond with the cyclic ortho-ester
n is an integer of between 1 and 9 inclusive
m is an integer of between 2 and 6 inclusive, it being possible for the values of m to be identical or different within the different groups —(CH)ₘ—NH—.

According to another alternative, this may also include a radical of the polyamine type of general formula: 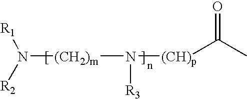
in which:
R₁, R₂, and R₃ represent, independently of each other, a hydrogen atom or a group —(CH₂)_{q}—NRR═, it being possible for q to vary between 1, 2, 3, 4, 5 and 6, this being in an independent manner between the different groups R₁, R₂ and R₃, and R and R═ representing, independently of each other, a hydrogen atom or a group —(CH₂)_{q=}—NH₂, it being possible for q= to vary between 1, 2, 3, 4, 5 and 6, this being in an independent manner between the different groups R and R═,
m and p represent, independently of each other, an integer which may vary between 1 and 6, and
n represents an integer which may vary between 0 and 6, with when n is greater than 1, it being possible for m to take different values and R₃ different meanings in the general formula, and with, when n is equal to 0, at least one of R₁ and R₂ which is different from hydrogen.

According to another aspect of the present invention, the radical of the polyamine type may also be represented by a substituent with a general formula identical to the preceding one, but with R and R═ representing, independently of each other, a hydrogen atom or a group of formula (1): 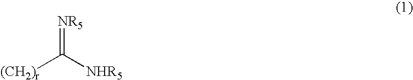
in which r is an integer which may vary from 0 to 6 inclusive, and the groups R₅ represent, independently of each other, a hydrogen atom, an alkyl, carbamate or aliphatic or aromatic acyl substituent, which is optionally halogenated, it being understood that at least one of the groups R₁, R₂ and R₃ comprises at least one group of formula (1).

A radical of the polyamine type is thus obtained which comprises one or more terminal guanidine functions.

According to another variant of the invention, the radical of the polyamine type may also represent a polyamine such as those described above but with a terminal group of the cyclic guanidine type (instead of an amine or a guanidine) of general formula (2): 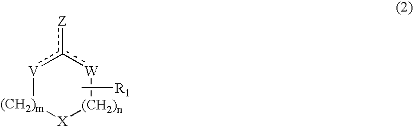
for which:
m and n are integers, independent of each other, of between 0 and 3 inclusive and such that m+n is greater than or equal to 1,
R₁ represents a group of general formula (3):
   (CH₂)ₚ—Y_{q}(*) (3)
   for which p and q are integers, independent of each other, of between 0 and 10 inclusive, Y represents a carbonyl, amino, methylamino or alternatively methylene group, it being possible for Y to have different meanings in the different groups [(CH₂)ₚ—Y], and (*) represents either a hydrogen atom or a covalent bond, it being understood that R₁ may be linked to any atom of the general formula (2), including Z, and that there is a single group R₁ in the formula (2),
X represents a group NR₂ or alternatively CHR₂, R₂ being either a hydrogen atom or the bond with the group R₁ as defined above,
the group 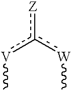
   represents:
1st case: a group of general formula (4): 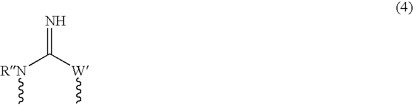
   for which W═ represents CHR═══ or alternatively NR═══, and R══ and R═══ represent, independently of each other, a hydrogen atom, a methyl, or the bond with the group R₁ as defined above, or alternatively
2nd case: a group of general formula (5): 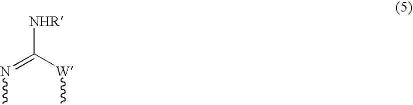
   for which W═ represents CHR═══ or alternatively NR═══, and R═ and R═══ represent, independently of each other, a hydrogen atom, a methyl, or the bond with the group R₁ as defined above.

In general, any other radical of the polyamine type known to persons skilled in the art for combining with nucleic acids, in particular via electrostatic interactions, may also be suitable.

For the purposes of the present invention, there is understood by single- or double-chain alkyls the hydrophobic radicals consisting of one or two linear alkyl chains comprising 10 to 24 carbon atoms and optionally comprising one or more unsaturations. In the case of the double-chain alkyls, this may include for example two alkyl chains bonded to a nitrogen atom so as to form a dialkylamino substituent the two alkyl chains being, for example, linear and comprising 10 to 24 carbon atoms and optionally one or more unsaturations. This may also include saturated or unsaturated fatty acids such as, for example palmitic acid, oleic acid, stearic acid or alternatively myristic acid. Preferably, the single- or double-chain alkyls possess 12 to 18 carbon atoms, and more preferably still, they are chosen from the groups possessing 12, 14, 16 or 18 carbon atoms (for each alkyl chain).

There is understood by “steroid derivative” for the purposes of the present invention the substituents chosen for example from sterols, steroids and steroid hormones. More preferably, the steroid derivatives are chosen from cholesterol, cholestanol, 3-αa-5-cyclo-5-αa-cholestan-6-βb-ol, cholic acid, cholesteryl formate, cholestanyl formate, 3αa,5-cyclo-5αa-cholestan-6βb-yl formate, cholesterylamine, 6-(1,5-dimethylhexyl)-3a,5a-dimethylhexadecahydrocyclopenta-[a]cyclopropa[2,3]cyclopenta[1,2-f]naphthalen-10-yl-amine, cholestanylamine or alternatively dexamethasone.

The hydrophobic dendrimers according to the present invention are preferably chosen from hydrophobic poly(alkyl ethers) or alternatively hydrophobic poly(aryl ethers). In a particularly advantageous manner, the hydrophobic dendrimers according to the present invention are chosen from poly(benzyl ethers).

For the purposes of the present invention, the polyalkylene glycols are preferably chosen from polyalkylene glycols having an average molecular weight of between 10² and 10⁵ Daltons (Da), and optionally covalently linked to a targeting element. In a particularly advantageous manner, the polyalkylene glycols according to the present invention are chosen from polyethylene glycols (PEG) having an average molecular weight of between 10² and 10⁵ Da, and more preferably between 500 and 10⁵ Da.

For the purposes of the present invention, there is understood by “mono- or polysaccharide” the molecules consisting of one or more saccharides, optionally covalently linked to a targeting element. There may be mentioned by way of example pyranoses and furanoses, for example glucose, mannose, rhamnose, galactose, fructose or alternatively maltose, lactose, saccharose, sucrose, fucose, cellobiose, allose, laminarobiose, gentiobiose, sophorose, melibiose and the like. Furthermore, this may also include so-called “complex” saccharides, that is to say several saccharides which are covalently coupled to each other, each sugar being preferably chosen from the list cited above. By way of suitable polysaccharides, there may be mentioned dextrans, α-amylose, amylopectin, fructans, mannans, xylans and arabinans. Preferably, the mono- or polysaccharides according to the present invention are chosen from natural or commercial derivatives which are compatible with pharmacological applications such as natural sugars, cyclodextrins or alternatively dextrans.

According to another alternative of the present invention, the polyalkylene glycol or the mono- or polysaccharide may optionally be covalently linked to a targeting element. In this case, this may include either an extracellular targeting element which makes it possible to orient the acid-sensitive compounds according to the present invention or the compositions containing them toward certain cell types or certain desired tissues (tumor cells, hepatic cells, hematopoietic cells and the like), or alternatively this may include an intracellular targeting element which allows orientation toward certain preferred cellular compartments (mitochondria, nucleus and the like).

Among the targeting elements which can be used in the context of the invention, there may be mentioned sugars, peptides, proteins, oligonucleotides, lipids, neuromediators, hormones, vitamins or derivatives thereof. Preferably, this includes sugars, peptides, vitamins or proteins such as for example antibodies or antibody fragments, ligands for cellular receptors or fragments thereof, receptors or alternatively receptor fragments. For example, this may include ligands for growth factor receptors, cytokine receptors, receptors of the cellular lectin type, folate receptors, or ligands having the sequence RGD with affinity for the receptors for adhesion proteins such as integrins. There may also be mentioned the receptors for transferrin, HDLs and LDLs, or the folate transporter. The targeting element may also be a sugar which makes it possible to target lectins such as the receptors for the asialoglycoproteins or for the syalydes such as Sialyl Lewis X, or alternatively an antibody Fab fragment, or a single-chain antibody (ScFv).

For the purposes of the present invention, there is understood by “polyalkyleneimines” the polymers described in the publication WO 96/02655, namely the polymers comprising the monomeric units of general formula: 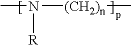
in which R may be a hydrogen atom or a group of formula: 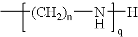
and n is an integer of between 2 and 10, p and q are integers chosen such that the sum p+q is such that the average molecular weight of the polymer is between 100 and 10⁷ Da.

It is understood that, in this formula, the value of n may vary between the different units —NR—(CH₂)ₙ—. Thus, this formula groups together both the homopolymers and the heteropolymers. Commercial polyalkyleneimines constitute an advantageous alternative. The polyethyleneimines (PEI) are most particularly preferred, and more specifically PEI 25K (PEI having an average molecular weight of 25 KDa), PEI 50K, PEI 100K or alternatively PEI 200K.

According to the present invention, there is understood by “therapeutic molecule” the molecules which make it possible to prevent or cure a pathology which manifests itself in the regions of the body producing an increased acidity compared with what is physiologically normal. Such regions are more specifically, but not solely:
tumors, in particular tumor cells and also normal cells in the vicinity of these tumors (for example the endothelial cells of the tumors), which exhibit a higher local acidity than what is physiologically normal (N. Raghunand et al., Drug Resistance Updates, 2000, 3, pp. 30-38),
muscles affected by ischemia, for example the cardiac muscle, in which the acidosis partly results from the lactic acid produced by the anaerobic fermentation of hydrocarbons of the sugar type or of the fatty acids,
the inflammation areas where the production of superoxide ions by the macrophages consumes a lot of oxygen,
or alternatively the tissues where a metabolic, infectious or inflammatory disorder produces local acidosis.

According to another alternative, the “therapeutic molecules” according to the present invention make it possible to prevent or cure a pathology by their release into an acidic cellular compartment, for example into the endosome of the cells which is acidic.

The therapeutic molecules may thus be chosen for example from peptides, oligopeptides, proteins, antigens and their antibodies, enzymes and their inhibitors, hormones, antibiotics, analgesics, bronchodilators, antimicrobials, antihypertensive agents, cardiovascular agents, agents acting on the central nervous system, antihistamines, antidepressants, tranquilizers, anticonvulsants, anti-inflammatory substances, stimulants, antiemetics, diuretics, antispasmodics, antiischemics, agents limiting cell death, or alternatively anticancer agents.

In addition, there is understood by “biologically active substance” the substances chosen either from the therapeutic molecules as defined above, or from nucleic acids.

For the purposes of the invention, there is understood by “nucleic acid” both a deoxyribonucleic acid and a ribonucleic acid. This may include natural or artificial sequences, and in particular genomic DNA (gDNA), complementary DNA (cDNA), messenger RNA (mRNA), transfer RNA (tRNA), ribosomal RNA (rRNA), hybrid sequences or synthetic or semisynthetic sequences, oligonucleotides which are modified or otherwise. These nucleic acids may be for example of human, animal, plant, bacterial, viral or alternatively synthetic origin. They may be obtained by any technique known to persons skilled in the art, and in particular by screening libraries, by chemical synthesis, or alternatively by mixed methods including the chemical or enzymatic modification of sequences obtained by screening libraries. They may be modified chemically.

As regards more particularly the deoxyribonucleic acids, they may be single or double-stranded as well as short oligonucleotides or longer sequences. In particular, the nucleic acids advantageously consist, for example, of plasmids, vectors, episomes or expression cassettes. These deoxyribonucleic acids may in particular carry a replication origin which is functional or otherwise in the target cell, one or more marker genes, sequences for regulation of transcription or of replication, genes of therapeutic interest, antisense sequences which are modified or otherwise, or alternatively regions for binding to other cellular components.

Preferably, the nucleic acid comprises an expression cassette consisting of one or more genes of therapeutic interest under the control of one or more promoters and of a transcriptional terminator which are active in the target cells.

For the purposes of the invention, there is understood by “expression cassette for a gene of interest” a DNA fragment which may be inserted into a vector at specific restriction sites. The DNA fragment comprises a nucleic acid sequence encoding an RNA or nucleic peptide of interest and comprises, in addition, the sequences necessary for the expression (activator(s), promoter(s), polyadenylation sequences and the like) of said sequence. The cassette and the restriction sites are designed to ensure insertion of the expression cassette into an appropriate reading frame for transcription and translation.

This generally includes a plasmid or an episome carrying one or more genes of therapeutic interest. By way of example, there may be mentioned the plasmids described in patent applications WO 96/26270 and WO 97/10343 incorporated into the present by reference.

For the purposes of the invention, there is understood by gene of therapeutic interest in particular any gene encoding a protein product having a therapeutic effect. The protein product thus encoded may be in particular a protein or a peptide. This protein product may be exogenous, homologous or endogenous with respect to the target cell, that is to say a product which is normally expressed in the target cell when the latter exhibits no pathology. In this case, the expression of a protein makes it possible, for example, to compensate for an inadequate expression in the cell or the expression of an inactive or a weakly active protein because of a modification, or alternatively to overexpress said protein. The gene of therapeutic interest may also encode a mutant of a cellular protein, which has for example increased stability or a modified activity. The protein product may also be heterologous with respect to the target cell. In this case, an expressed protein can for example supplement or provide an activity which is deficient in the cell, allowing it to combat a pathology, or to stimulate an immune response.

Among the therapeutic products for the purposes of the present invention, there may be mentioned more particularly enzymes, blood derivatives, hormones, lymphokines, interleukins, interferons or TNF for example: FR 92/03120), growth factors, neurotransmitters or their precursors or synthesis enzymes, trophic factors (for example BDNF, CNTF, NGF, IGF, GMF, aFGF, bFGF, NT3, NT5, or alternatively HARP/pleiotrophin), apolipoproteins (for example ApoAI, ApoAIV, or ApoE: FR 93/05125), dystrophin or a minidystrophin (FR 91/11947), cystic fibrosis associated protein CFTR, tumor suppresser genes (for example p53, Rb, Rap1A, DCC, or k-rev: FR 93/04745), genes encoding factors involved in coagulation (factors VII, VIII, IX), genes involved in DNA repair, suicide genes (thymidine kinase, cytosine deaminase), the genes for hemoglobin or other protein carriers, metabolic enzymes, catabolic enzymes and the like.

The nucleic acid of therapeutic interest may also be a gene or an antisense sequence, whose expression in the target cell makes it possible to control the expression of genes or the transcription of cellular mRNAs. Such sequences can, for example, be transcribed in the target cell into RNAs which are complementary to cellular mRNAs and thus block their translation to protein, according to the technique described in patent EP 140 308. The therapeutic genes also comprise the sequences encoding ribozymes, which are capable of selectively destroying target RNAs (EP 321 201).

As indicated above, the nucleic acid may also comprise one or more genes encoding an antigenic peptide, capable of generating an immune response in humans or animals. In this particular embodiment, the invention allows the production either of vaccines or of immunotherapeutic treatments applied to humans or animals, in particular against microorganisms, viruses or cancer. This may include in particular antigenic peptides specific for the Epstein-Barr virus, the HIV virus, the hepatitis B virus (EP 185 573), the pseudo-rabies virus, the syncitia forming virus, other viruses or alternatively antigenic peptides specific for tumors (EP 259,212).

Preferably, the nucleic acid also comprises sequences allowing the expression of the gene of therapeutic interest and/or of the gene encoding the antigenic peptide in the desired cell or organ. This may include sequences which are naturally responsible for the expression of the gene considered when these sequences are capable of functioning in the infected cell. This may also include sequences of a different origin (responsible for the expression of other proteins, or even synthetic). In particular, this may include promoter sequences of eukaryotic or viral genes. For example, this may include promoter sequences derived from the genome of the cell which it is desired to infect. Likewise, this may include promoter sequences derived from the genome of a virus. In this regard, there may be mentioned for example the promoters of the E1A, MLP, CMV and RSV genes and the like. In addition, these expression sequences may be modified by addition of activating or regulatory sequences and the like. This may also include an inducible or repressible promoter.

Moreover, the nucleic acid may also comprise, in particular upstream of the therapeutic gene of interest, a signal sequence directing the therapeutic product synthesized in the secretory pathways of the target cell. This signal sequence may be the natural signal sequence of the therapeutic product, but it may also be any other functional signal sequence, or an artificial signal sequence. The nucleic acid may also comprise a signal sequence directing the therapeutic product synthesized toward a particular compartment of the cell.

According to a preferred aspect of the invention, the acid-sensitive compounds are more specifically chosen from the compounds of general formula: 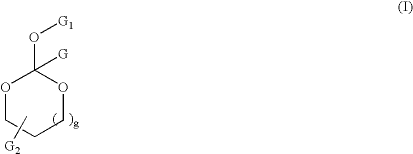
in which:
g is an integer equal to 0 or 1
G represents a substituent chosen from the hydrogen atom, the alkyl substituents comprising 1 to 6 carbon atoms in the form of a saturated or unsaturated, straight or branched chain, or aryls, and
G₁ and G₂ represent:
(a) one a hydrophilic substituent chosen from radicals of the polyamine type, and the other a hydrophobic substituent chosen from single- or double-chain alkyls or steroid derivatives, or alternatively
(b) one a hydrophobic linear alkyl group comprising 10 to 24 carbon atoms and optionally comprising one or more unsaturations, and the other a group of general formula: 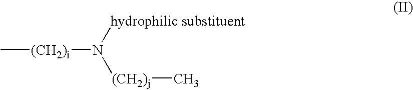
   in which i is an integer ranging from 1 to 4 and j is an integer ranging from 9 to 23, and the hydrophilic substituent is chosen from radicals of the polyamine type, or alternatively
(c) one a hydrophilic substituent chosen from polyalkylene glycols and the other a substituent chosen from polyalkyleneimines, or alternatively
(d) one a hydrophilic substituent chosen from polyalkylene glycols and the other a hydrophobic substituent chosen from single- or double-chain alkyls, steroid derivatives or the covalent conjugates between a single- or double-chain alkyl or a steroid derivative and a polyalkylene glycol molecule comprising 1 to 20 monomeric units, or alternatively
(e) one a hydrophilic substituent chosen from polyalkylene glycols and the other a therapeutic molecule.

More preferably still, the acid-sensitive compounds of the invention are chosen from the compounds of general formula: 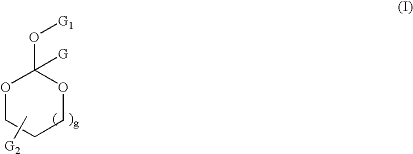
in which:
g is an integer equal to 0 or 1
G represents a substituent chosen from the hydrogen atom, the alkyl radicals comprising 1 to 6 carbon atoms in the form of a saturated or unsaturated, straight or branched chain, or phenyl, and
G₁ and G₂ represent:
(a) one a hydrophilic substituent chosen from the radicals of the polyamine type, and the other a hydrophobic substituent chosen from single- or double-chain alkyls or steroid derivatives, or alternatively
(d) one a hydrophilic substituent chosen from polyalkylene glycols and the other a hydrophobic substituent chosen from single- or double-chain alkyls or steroid derivatives.

The novel acid-sensitive compounds of general formula (I) may be provided in the form of nontoxic and pharmaceutically acceptable salts. These nontoxic salts comprise the salts with inorganic acids (hydrochloric, sulfuric, hydrobromic, phosphoric or nitric acids) or with organic acids (acetic, propionic, succinic, maleic, hydroxymaleic, benzoic, fumaric, methanesulfonic or oxalic acids).

The acid-sensitive compounds according to the present invention may be prepared according to many methods chosen from those described in the literature for the synthesis of molecules containing a cyclic ortho-ester group (for example, reference may be made to the examples given in the review Synthesis, Robert H. DeWolfe, 1974, pp. 153-172). According to an alternative which can be envisaged, the acid-sensitive compounds of general formula (I) may for example be obtained by reacting an alcohol of formula G₁OH with an ortho-ester of general formula: 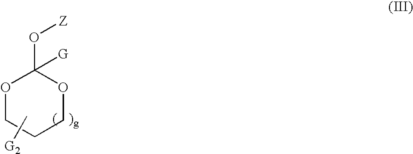
in which g, G, G₁ and G₂ are as defined for the general formula (I), and Z represents a linear or branched alkyl group containing 1 to 4 carbon atoms.

This substitution may be carried out in the presence of an acid catalyst and/or may be heat activated at a temperature of between 50 and 150° C., with or without solvent. If it is chosen to carry out the procedure in the presence of a solvent, the latter is chosen from conventional organic chemistry solvents such as for example the organochlorinated solvents, aromatic solvents or alternatively ethers. When a catalyst is used, it may be an inorganic or organic acid, a Lewis or Brönsted acid. For example, the catalyst may be chosen from hydrochloric acid, sulfuric acid, para-toluenesulfonic acid, camphorsulfonic acid, pyridinium para-toluenesulfonate, or alternatively magnesium chloride. This substitution can also be favored by distilling the alcohol ZOH produced during the reaction if it is more volatile than the alcohol G₁OH. This continuous distillation may be carried out by heating at atmospheric pressure or under reduced pressure.

The starting alcohol G₁OH is either commercially available, or it can be synthesized by any method known to persons skilled in the art, for example by hydration of the corresponding alkene, by hydrolysis of the corresponding halogenated derivative, or alternatively by reducing the corresponding carbonyl-containing derivative.

According to another variant of the invention, the group Z may already represent the group G₁ and in this case, the step for the reaction between the ortho-ester of general formula (III) and the alcohol G₁OH is not necessary.

The compound of general formula (III) may be obtained by the action of a trialkyl ortho-ester of general formula (IV): 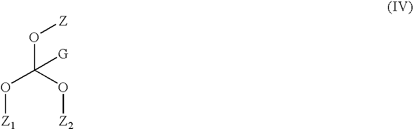
in which Z and G are as defined above, and Z₁ and Z₂, which are identical or different, represent linear or branched alkyl groups containing 1 to 4 carbon atoms, on a diol of general formula (V): 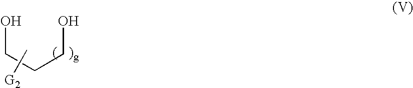
in which g and G₂ are as defined above.

The reaction may be carried out according to conventional methods for protecting diols as ortho-ester, for example according to the methods indicated by T. W. Greene and P. G. M. Wuts in “Protective Groups in Organic Synthesis” (2^{nd} Ed., Wiley-Interscience, pp. 135-136). The procedure is generally carried out in a conventional organic solvent (for example organochlorinated solvents, aromatic solvents, ethers and the like) in the presence of an acid catalyst. The catalyst may be chosen from inorganic or organic acids, Lewis or Brönsted acids. For example, it is possible to use hydrochloric acid, sulfuric acid, para-toluenesulfonic acid, camphorsulfonic acid, pyridinium para-toluenesulfonate, or alternatively magnesium chloride.

The trialkyl ortho-ester of general formula (IV) is either commercially available, or it can be synthesized according to conventional methods known to persons skilled in the art, for example from the corresponding ester, or alternatively by substitution of the alkoxy groups starting with another commercial trialkyl ortho-ester.

The diol of general formula (V) is either commercially available, or can be obtained by the reaction between a commercial diol and G₂, or alternatively it can be obtained by direct functionalization of G₂ to a diol. This functionalization may for example consist in an oxidation of the corresponding alkene, or alternatively in the opening of a corresponding epoxide, according to methodologies well known to persons skilled in the art.

When either of G₁ and G₂ represents a radical of the polyamine type, it is either commercially available, or it is obtained according to conventional methods known to persons skilled in the art, for example according to the methods described in the prior art (for example in the publications WO 96/17823, WO 97/18185, WO 98/54130 or alternatively WO 99/51581), or according to analogous methods.

When either of G₁ and G₂ represents a hydrophobic substituent chosen from the single- or double-chain alkyls, the latter is either commercially available, or it is obtained according to conventional methods known to persons skilled in the art. For example, when this includes a dialkylamino substituent with a long carbon chain, it can be prepared from the corresponding primary amine by alkylation (monosubstitution of a halogenated alkyl), by alkylative reduction (from an aldehyde), or alternatively by condensation/reduction (formation of an amide function from an acid and then reduction).

When either of G₁ and G₂ represents a hydrophobic substituent chosen from the steroid derivatives or the hydrophobic dendrimers, it is preferably chosen from commercially available products.

When either of G₁ and G₂ represents a substituent chosen from polyalkylene glycols or mono- or polysaccharides, the latter is either commercially available, or it is obtained by conventional methods known to persons skilled in the art, in particular by polymerization. In the case or this substituent is covalently linked to a targeting element, the synthesis of the acid-sensitive compounds according to the present invention described above can be carried out before or after the binding, by the conventional methods of persons skilled in the art, of said targeting element to this substituent.

When either of G₁ and G₂ represents a substituent chosen from polyalkyleneimines, the latter is either commercially available, or it is obtained according to the conventional methods known to a person skilled in the art or according to the methods described in the prior art, for example in the publication WO 96/02655.

The method of preparation indicated above constitutes only a method given by way of illustration, and any other equivalent method of preparation can naturally also be used. For example, it is possible to carry out the reactions starting with a diol of general formula (V) which does not possess the group G₂ but, in place, a functional group which is optionally protected (for example a protected amine), by carrying out an additional final step for the binding of the group G₂ (for example, deprotection of the amine and then condensation of the acid of formula G₂COOH).

Another subject of the invention relates to the compositions comprising at least one acid-sensitive compound of general formula (I) as defined above. According to a variant of the invention, said compositions comprise at least one biologically active substance and an acid-sensitive compound of general formula (I) in which G₁ and G₂ have the definitions indicated under (a), (b), (c) or (d).

The compositions according to the invention may, in addition, comprise one or more adjuvants capable of binding with the complexes formed between the acid-sensitive compound according to the invention and the biologically active substance. In another embodiment, the present invention therefore relates to the compositions comprising at least one biologically active substance, an acid-sensitive compound of formula (I) in which G₁ and G₂ have the definitions indicated under (a), (b), (c) or (d), and one or more adjuvants. The presence of this type of adjuvants (lipids, peptides or proteins for example) can advantageously make it possible to increase the transfecting power of the compounds in the cases where the biologically active substance is a nucleic acid to be transfected.

In this perspective, the compositions according to the present invention may comprise, as adjuvant, one or more neutral lipids. It has indeed been shown that the addition of a neutral lipid makes it possible to improve the formation of the nucleolipid particles (in the case where the biologically active substance is a nucleic acid), and to promote the penetration of the particle into the cell by destabilizing its membrane.

More preferably, said neutral lipids are lipids with two fatty chains. In a particularly advantageous manner, natural or synthetic, zwitterionic lipids or lipids free of ionic charge under physiological conditions, are used. They may be chosen more particularly from dioleoylphosphatidylethanolamine (DOPE), oleoylpalmitoylphosphatidylethanolamine (POPE), distearoylphosphatidylethanolamine, dipalmitoyl-phosphatidylethanolamine, dimirystoylphosphatidyl-ethanolamine as well as their derivatives which are N-methylated 1 to 3 times, phosphatidylglycerols, diacylglycerols, glycosyldiacylglycerols, cerebrosides (such as in particular galactocerebrosides), sphingolipids (such as in particular sphingomyelins) or alternatively asialogangliosides (such as in particular asialoGM1 and GM2).

These various lipids may be obtained either by synthesis, or by extraction from organs (example: the brain) or from eggs, by conventional techniques well known to persons skilled in the art. In particular, the extraction of the natural lipids may be carried out by means of organic solvents (see also Lehninger, Biochemistry).

Preferably, in the case where the biologically active substance is a nucleic acid, the compositions of the invention comprise from 0.01 to 20 equivalents of adjuvant(s) for one equivalent of nucleic acids in mol/mol and, more preferably, from 0.5 to 5.

The acid-sensitive compounds according to the invention may have various uses depending on the substituents G₁ and G₂ situated on either side of the cyclic ortho-ester.

In the case where the substituents G₁ and G₂ have the definitions indicated in (a), (b) or (c) in the general formula (I), the acid-sensitive compounds according to the invention can form conjugates (for example of the type including liposomes, complexes or alternatively nanoparticles) directly with biologically active substances which may then be released into the tissues or cellular compartments, which are more acidic than what is physiologically normal. These acid-sensitive compounds are in particular more particularly useful for the transfection of nucleic acids.

In the case where the substituents G₁ and G₂ have the definitions indicated in (d) in the general formula (I), the acid-sensitive compounds according to the invention constitute nonionic surfactants which make it possible both to stabilize particles encapsulating a biologically active substance and to release said biologically active substance by degradation in the regions which are very weakly acidic to acidic in the body, in particular regions where the pH is acidic and is between about 4 and about 7.

In addition, the polysaccharide or polyalkylene glycol substituents, and more specifically polyethylene glycol (PEG), are known to confer a sort of “furtiveness” on the particles with which they are associated by inhibiting the nonspecific adsorption by the serum proteins, and consequently the recognition of said particles by the microphages (see for example Torchilin et al., Biochim. Biophys. Acta 1994, 1195, pp. 11-20 or Papahadjopoulos et al., PNAS 1991, 88, p. 11460-4). Thus, the acid-sensitive compounds comprising a PEG molecule according to the invention have an advantage from the safety point of view and also an additional advantage in the sense that they reduce the risk of interference with other proteins. At the level of the acidic regions in the body, the degradation of the ortho-ester present in the compounds according to the invention allows the separation of the PEG molecules from the rest of the particle, making the biologically active substance again “available” (there is in fact “disappearance of the furtiveness”). A selective transfer can thus be expected with respect to the acidic tissues.

Finally, in the case where the substituents G₁ and G₂ have the definitions indicated in (e) or (f) in the general formula (I), the acid-sensitive compounds according to the invention constitute covalent conjugates with a therapeutic molecule, thereby allowing its vectorization and then its release in the acidic regions of the body. These covalent conjugates are of the same type as those described by Kratz et al., but with a novel acid-sensitive bond between the therapeutic molecule and the “vector” part which has the advantage of having a modulable sensitivity compared with the pH-sensitive bonds used up until now.

Thus, the subject of the present invention is also the use of the acid-sensitive compounds of general formula (I) as defined above for the manufacture of a medicament intended for treating diseases. In this case, the disease targeted determines the choice of the biologically active substance.

According to a particularly advantageous variant, when the biologically active substance is a nucleic acid, the acid-sensitive compounds of general formula (I) in which G₁ and G₂ have the definitions indicated under (a), (b) or (c) can be used for the manufacture of a medicament intended for the in vitro, ex vivo or in vivo transfection of nucleic acids, in particular into primary cells or into established lines. This may include for example fibroblast cells, muscle cells, nerve cells (neurones, astrocytes, glyal cells), hepatic cells, cells of the hematopoietic line (lymphocytes, CD34, dendritic cells and the like), or alternatively epithelial cells, in differentiated or pluripotent form (precursors).

Finally, according to another alternative of the invention, the acid-sensitive compounds of general formula (I) in which G₁ and G₂ have the definitions indicated under (e) or (f) can be used as a medicament.

In the preceding text, the acid-sensitive compounds according to the present invention become degraded in the tissues or cellular compartments whose pH is more acidic than what is physiologically normal. However, according to another alternative, it is possible to induce or to increase the acidity in the target region of the body by a general or local treatment known to persons skilled in the art. There may be mentioned by way of example, without limitation, the injection of an acidic product into the region to be treated or alternatively the intravenous injection of glucose which causes specific acidification of tumor tissues (T. Volk et al.; Br. J. Cancer; 1993, 68 (3), 492-500). Thus, the acid-sensitive compounds according to the present invention may also be used in regions of the body which are a priori nonacidic and which have been made acidic by treatments known to persons skilled in the art.

For all the uses of the acid-sensitive compounds according to the present invention indicated above, the compositions according to the invention comprising:
either an acid-sensitive compound of general formula (I) in which G₁ and G₂ have the definitions indicated under (e) or (f),
or an acid-sensitive compound of general formula (I) in which G₁ and G₂ have the definitions indicated under (a), (b), (c) or (d) and a biologically active substance,
   can be formulated for administration, for example, by the topical, cutaneous, oral, rectal, vaginal, parenteral, intranasal, intravenous, intramuscular, subcutaneous, intraocular, transdermal, intratracheal or intraperitoneal route. Preferably, the compositions of the invention contain a pharmaceutically acceptable vehicle for an injectable formulation, in particular for a direct injection into the desired organ, or for administration by the topical route (to the skin and/or mucous membrane). This may include in particular isotonic, sterile solutions or dry, in particular freeze-dried compositions which, upon addition, depending on the case, of sterilized water or physiological saline, allow the preparation of injectable solutions. The doses of biologically active substances used for the injection as well as the number of administrations may be adjusted according to various parameters, and in particular according to the mode of administration used, the relevant pathology, the chain to be expressed when the biologically active substance is a nucleic acid, or alternatively the desired duration of treatment. As regards more particularly the mode of administration, this may include either a direct injection into the tissues, for example at the level of the tumors, or the circulatory pathways, or a treatment of cells in culture followed by their reimplantation in vivo, by injection or transplantation. The relevant tissues in the context of the present invention are for example the muscles, the skin, the brain, the lungs, the liver, the spleen, the bone marrow, the thymus, the heart, the lymph, blood, the bones, the cartilages, the pancreas, the kidneys, the bladder, the stomach, the intestines, the testicles, the ovaries, the rectum, the nervous system, the eyes, the glands or alternatively the connective tissues.

In addition to the preceding arrangements, the present invention also comprises other characteristics and advantages which will emerge from the examples and figures which follow, and which should be considered as illustrating the invention without limiting the scope thereof. In particular, the Applicant proposes, without limitation, various operating protocols as well as reaction intermediates which can be used to prepare the compounds of general formula (I). Of course, it is within the capability of persons skilled in the art to draw inspiration from these protocols and/or intermediate products in order to develop similar procedures so as to arrive at other compounds of general formula (I) according to the invention.

### FIGURES

FIG. 1: Variation of the level of fluorescence as a function of time at pH 5 of complexes formed between DNA and a control cationic lipid or alternatively the acid-sensitive compounds A Syn or Trans, in 3 different ratios: 0.4 or 1.7 or 6.0 nmol of cationic lipid or of acid-sensitive compound/μg of DNA.

FIG. 2: Efficiency of transfection in vitro into HeLa cells of complexes formed between DNA and compound A Syn or Trans or a control cationic lipid, at different charge ratios, with or without serum.

The y-axis represents the expression of luciferase in pg/well/μg of protein. The x-axis indicates the compound A Syn or Trans or control cationic lipid/DNA charge ratio.

FIG. 3: Variation of the size (in nm) of control cationic lipid/DNA nucleolipid particles as a function of the quantity of compound C or compound D or of Brij 700 or of a non-acid-sensitive analog of compound D (Analog D) used relative to the quantity of DNA (weight/weight). A small size indicates that the nucleolipid particles are stabilized. A very large size indicates on the contrary destabilization of the nucleolipid particles which then tend to aggregate.

FIG. 4: Variation of the size (in nm) of control cationic lipid/DNA/compound or analog D nucleolipid particles as a function of time, at different ratios (weight/weight), when the pH is 5. A small size of the nucleolipid particles indicates that they are stabilized. A very large size indicates on the contrary destabilization of the nucleolipid particles which then tend to aggregate.

FIG. 5: Variation of the size (in nm) of control cationic lipid/DNA/compound C or compound E nucleolipid particles as a function of time, at various pH values (pH 4, pH 5, pH 6 and pH 7.4). The compound C or compound E/DNA ratio is set at 1 (in nmol/μg of DNA).

FIG. 6: Schematic representation of the plasmid pXL3031.

FIG. 7: Dose/response curve for compound D on the in vivo gene transfer activity mediated by the cationic lipid/DOPE/DNA (5/5/1) complexes. The compound “Analog D” is used as a negative control. The data are averages (lines) and individual values (dots) for 4 Balb/c mice caryying subcutaneous M109 tumors.

### EXAMPLES

The usual reagents and catalysts such as triethylamine, trifluoroacetic acid, trifluoroacetic anhydride, tert-butyl bromoacetate, butyrolactone, 3-aminopropan-1,2-diol, serinol (2-aminopropan-1,3-diol), trimethyl ortho-formate, trimethyl ortho-acetate, para-toluenesulfonic acid, pyridinium para-toluenesulfonate or alternatively benzotriazol-1-yloxytris(dimethyl-amino)phosphonium (BOP) hexafluorophosphate, are commercially available.

The washings are performed with aqueous solutions saturated with sodium chloride, saturated with sodium hydrogen carbonate and with a concentrated solution of potassium hydrogen sulfate at 0.5 mol/l.

The hydrophilic polymers (polyethylene glycols of different sizes) are commercially available. The hydrophilic substituents of the polyamine type are also commercially available or alternatively they can be synthesized by conventional methods known to a person skilled in the art as indicated in particular in the examples which follow. The hydrophobic substituents (single- or double-chain dialkylamines, fatty alcohols and the like) are commercially available or alternatively synthesized according to conventional methods known to a person skilled in the art. For example, the single- or double-chain dialkylamines may be synthesized from primary amines and the corresponding halogenated alkyl derivatives as indicated in the examples which follow.

The Proton Nuclear Magnetic Resonance (¹H NMR) spectra were recorded on Bruker 300, 400 and 600 MHz spectrometers. The clinical shifts are expressed in ppm (part per million) and the multiplicities by the usual abbreviations.

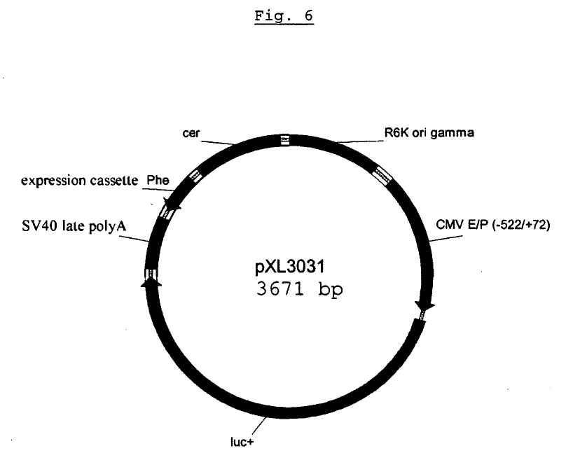

The plasmid used is pXL3031 described in the publication Gene Therapy (1999) 6, pp, 1482-1488, which contains the luc gene encoding luciferase under the control of the cytomegalovirus CMV E/P promoter. This plasmid is represented in FIG. 6. Its size is 3671 bp. The plasmid solution used is diluted to 1.262 g/l in water for injection.

### Example 1

### Synthesis of 2,2,2-trifluoro-N-(2-methoxy-[1,3]dioxolan-4-ylmethyl)acetamide (“Ortho 1”)

2,2,2-Trifluoro-N-(2-methoxy-[1,3]dioxolan-4-ylmethyl)acetamide (“Ortho 1”) Has the Formula:

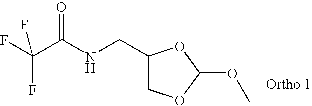

It can be obtained in two steps from 3-aminopropan-1,2-diol:

### 1) Preparation of N-(2,3-dihydroxypropyl)-2,2,2-trifluoroacetamide

15 g of 3-aminopropan-1,2-diol (164.6 mmol) are solubilized in 100 ml of tetrahydrofuran in a round-bottomed flask provided with a magnetic bar. The reaction mixture is then cooled to 0° C. on an ice bath and 21.5 ml of ethyl trifluoroacetate (181.1 mmol) are gradually added. The reaction mixture is stirred for 2 hours at room temperature. The crude reaction product is then evaporated to dryness. 29 g of a pure colorless oil are thus obtained (yield: 95%), which product is used without further purification.

### 2) Preparation of 2,2,2-trifluoro-N-(2-methoxy-[1,3]dioxolan-4-ylmethyl)acetamide (ortho 1)

The 29 g of N-(2,3-dihydroxypropyl)-2,2,2-trifluoroacetamide obtained in the preceding step (155 mmol) are solubilized in 75 ml of dichloromethane supplemented with 75 ml of trimethyl ortho-formate (685 mmol). 300 mg of para-toluenesulfonic acid (1.7 mmol) are then added and the reaction mixture is stirred for 2 hours at room temperature.

This crude product is then diluted in 500 ml of dichloromethane, washed with 3 times 200 ml of saturated sodium hydrogen carbonate and then 3 times 200 ml of saturated sodium chloride. The organic phase is dried over magnesium sulfate, filtered and concentrated to dryness. 30 g of a pure oily product are thus obtained (yield: 85%) without further purification.

¹H NMR (300 MHz, CDCl₃, δd in ppm) . A mixture of two diastereoisomers in the proportions 50/50 is observed.

* 3.33 and 3.37 (2s: 3H in total); from 3.35 to 3.80 (mts: 3H); from 4.10 to 4.25 (mt: 1H); 4.50 (mt: 1H); 5.73 and 5.78 (2s: 1H in total); 6.66 and 7.55 (2 unresolved complexes: 1H in total).

### Example 2

### Synthesis of 2,2,2-trifluoro-N-(2-methoxy-2-methyl-[1,3]dioxan-5-yl)acetamide (“Ortho 2)

2,2,2-Trifluoro-N-(2-methoxy-2-methyl-[1,3]dioxan-5-yl)acetamide (“Ortho 2”) has the formula:

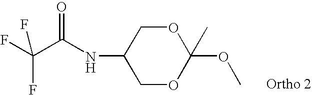

It can be obtained in two steps from 2-aminopropan-1,3-diol (serinol):

### 1) Preparation of 2,2,2-trifluoro-N-(2-hydroxy-1-hydroxymethylethyl)acetamide

4 g of 2-aminopropan-1,3-diol (43.9 mmol) are supplemented with 20 ml of tetrahydrofuran. The reaction mixture is then cooled to 0° C. on an ice bath and 5.8 ml of ethyl trifluoroacetate (48.3 mmol) are gradually added. This solution is stirred for 2 hours at room temperature.

The reaction mixture is then evaporated to dryness, taken up 3 times in dichloromethane so as to completely evaporate the tetrahydrofuran. 8.1 g of white powder (yield: 99%) are obtained pure and used in the next step without further purification.

### 2) Preparation of 2,2,2-trifluoro-N-(2-methoxy-2-methyl-[1,3]dioxan-5-yl)acetamide (Ortho 2)

7.9 g of 2,2,2-trifluoro-N-(2-hydroxy-1-hydroxymethylethyl)acetamide obtained in the preceding step (42.2 mmol) are solubilized in 30 ml of dichloromethane supplemented with 16.1 ml of trimethyl ortho-acetate (126.7 mmol). 73 mg of para-toluenesulfonic acid (0.42 mmol) are then added and the reaction mixture is stirred for 3 hours at room temperature.

The crude reaction product is then diluted with 150 ml of dichloromethane, washed with 3 times 50 ml of a saturated sodium hydrogen carbonate solution, and then 3 times 50 ml of a saturated sodium chloride solution. The organic phase is dried over magnesium sulphate, filtered and concentrated to dryness. 9.9 g of a white solid are obtained pure without further purification (yield: 96%).

¹H NMR (300 MHz, CDCl₃, δd in ppm). A mixture of two diastereoisomers in the approximate proportions 75/25 is observed.

* 1.49 and 1.50 (2 s: 3H in total); 3.34 and 3.35 (2 s: 3H in total); 3.66 and 3.82 (respectively dmt, J=12 Hz and dd, J=11 and 8 Hz: 2H in total); from 3.90 to 4.00 and from 4.20 to 4.35 (2 mts: 1H in total); 3.97 and 4.33 (respectively dd, J=11 and 5 Hz and dmt, J=12 Hz: 2H in total); 6.38 and 7.04 (2 broad unresolved complexes: 1H in total).

### Example 3

### Synthesis of the Syn and Trans Compounds 4-{4-[(2-{3-[4-(3-amino-propylamino)butylamino]propyl-amino}acetylamino)methyl]-[1,3]dioxolan-2-yloxy}-N,N-dioctadecylbutyramide tetraacetate

This example describes the route for the synthesis of the acid-sensitive compound A in the form of its two distinct diastereoisomeric forms Syn and Trans of formula:}

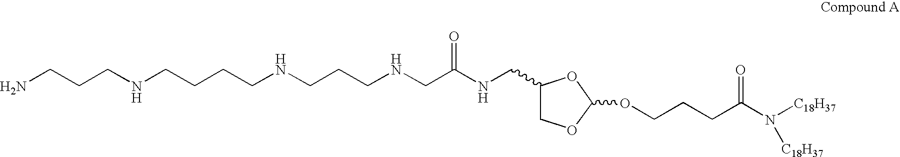

### a—Synthesis of the Syn and Trans 4-(4-aminomethyl-[1,3]dioxolan-2-yloxy)-N,N-dioctadecylbutyramide (lipid portion-O-ortho 1-NH₂)

This synthesis is carried out in three stages: functionalization of the dioctadecylamine to an alcohol and attachment onto the group Ortho 1 whose protecting group is then cleaved.

### 1) Preparation of 4-hydroxy-N,N-dioctadecylbutyramide

4.6 g of aluminium chloride (34 mmol) are supplemented with 25 ml of chloroform, the whole being cooled to about 10° C. on a thermostated bath. 6.4 ml of triethylamine (46 mmol) in 15 ml of chloroform are added dropwise and then the reaction mixture is allowed to return to room temperature. 6 g of dioctadecylamine (11.5 mmol), mixed with 1 ml of butyrolactone (13.8 mmol) in 110 ml of chloroform, are gradually added to the mixture using a dropping funnel.

The reaction no longer changes after magnetical stirring for 2 hours at room temperature. 75 ml of water are then added and the reaction mixture is stirred for 30 minutes. The crude product is filtered on Celite and then washed with chloroform. The filtrate is separated by settling out and the organic phase is washed with 3 times 50 ml of a saturated sodium chloride solution. The chloroformic solution is dried over magnesium sulfate, filtered and concentrated. 4.9 g of a white powder are obtained after chromatography on silica (yield: 70%).

### 2) Preparation of the Syn and Trans N,N-dioctadecyl-4-{4-[2,2,2-trifluoroacetylamino)methyl]-[1,3]dioxolan-2-yloxy}butyramides

2.8 g of 4-hydroxy-N,N-dioctadecylbutyramide obtained in the preceding step (4.6 mmol) are mixed with 2.6 g of 2,2,2-trifluoro-N-(2-methoxy-[1,3]dioxolan-4-ylmethyl)acetamide (Ortho 1, 11.5 mmol) and 90 mg of magnesium chloride (0.92 mmol). The whole is heated without solvent at 80° C. for two hours.

The crude reaction product is then dissolved in 150 ml of cyclohexane and washed with 3 times 30 ml of a saturated sodium hydrogen carbonate solution, and then with 3 times 30 ml of a saturated sodium chloride solution. The organic phase is dried over magnesium sulfate, filtered and concentrated. The purification is carried out by chromatography on silica. 1.2 g and 1.1 g of the two expected diastereoisomers Syn and Trans are thus isolated in the form of a white powder (yield: 62%).

¹H NMR for the compound SYN (300 MHz, CDCl₃, δ in ppm): 0.89 (t, J=7 Hz: 6H); from 1.15 to 1.40 (mt: 60H); 1.52 (mt: 4H); 1.94 (mt: 2H); 2.39 (t, J=7 Hz: 2H); 3.20 (broad t, J=8 Hz: 2H); 3.29 (mt: 2H); 3.61 (t, J=5 Hz: 2H); 3.66 (mt: 2H); 3.79 (dd, J=8 and 7 Hz: 1H); 4.11 (t, J=8 Hz: 1H); 4.50 (mt: 1H); 5.82 (s: 1H); 8.13 (unresolved complex: 1H).

¹H NMR for the compound TRANS (300 MHz, CDCl₃, δ in ppm): 0.89 (t, J=7 Hz: 6H); from 1.15 to 1.40 (mt: 60H); 1.52 (mt: 4H); 1.95 (mt: 2H); 2.38 (t, J=7 Hz: 2H); 3.21 (broad t, J=8 Hz: 2H); 3.29 (broad t, J=8 Hz: 2H); 3.44 (mt, 1H); from 3.55 to 3.75 (mt: 1H); 3.60 (t, J=6 Hz: 2H); 3.69 (dd, J=8.5 and 5 Hz: 1H); 4.19 (dd, J=8.5 and 7 Hz: 1H); 4.50 (mt: 1H); 5.87 (s, 1H); 6.70 (unresolved complex: 1H).

### 3) Preparation of the Syn and Trans 4-(4-aminomethyl-[1,3]dioxolan-2-yloxy)-N,N-dioctadecylbutyramide

1.1 g of N,N-dioctadecyl-4-{4-[(2,2,2-trifluoroacetylamino)methyl]-[1,3]dioxolan-2-yloxy}butyramide obtained in the preceding step (1.37 mmol, Syn or Trans) are dissolved in 10 ml of tetrahydrofuran and 10 ml of molar sodium hydroxide at 4% are added, with vigorous stirring. The reaction is left overnight at room temperature.

The tetrahydrofuran is then concentrated and then the product is extracted with three times 150 ml of diethyl ether. The organic phase is dried over calcium chloride, filtered and evaporated. 840 mg of the expected products are thus isolated (yield: 86%), and used as they are for the next step.

### b—Synthesis of (trifluoroacetyl-{3-[trifluoroacetyl-(4-{trifluoroacetyl-[3-(2,2,2-trifluoroacetylamino)-propyl]amino}butyl)amino]propyl}amino)acetic acid (hydrophilic part polyamine-COOH)

This synthesis is performed in two steps: protection of the four amines of the spermine and then substitution of one of the primary amines with the protected bromoacetic acid.

### 1) Synthesis of 2,2,2-trifluoro-N-[3-(2,2,2-trifluoroacetylamino)propyl]-N-(4-{trifluoroacetyl-[3-(2,2,2-trifluoroacetylamino)propyl]amino}-butyl)acetamide

8 g of spermine (39.5 mmol) are solubilized in 75 ml of dichloromethane. 33 ml of triethylamine (237 mmol) are added and then the reaction mixture is cooled to 0° C. on an ice bath. 41.5 g of trifluoroacetic anhydride diluted in 100 ml of dichloromethane are then added dropwise over 1 hour using a dropping funnel. The reaction mixture is then allowed to return to room temperature and the reaction is left overnight with stirring.

75 ml of a 5% sodium hydrogen carbonate solution are then added to the reaction mixture and the solution is stirred for 15 minutes at room temperature. The aqueous phase is extracted with 3 times 150 ml of dichloromethane. The organic phases are combined and washed with 3 times 100 ml of a concentrated potassium hydrogen sulfate solution at 0.5 M, and then 3 times 100 ml of a saturated sodium chloride solution. The organic phase is then dried over magnesium sulfate, filtered and concentrated to dryness. 22.5 g of a pure yellow powder are isolated without further purification (yield: 97%).

### 2) Preparation of (trifluoroacetyl-{3-[trifluoroacetyl-(4-{trifluoroacetyl-[3-(2,2,2-trifluoroacetylamino)-propyl]amino}butyl)amino]propyl}amino)acetic acid

1 g of sodium hydride (60% in oil, that is 25.6 mmol) are supplemented with 60 ml of dry dimethylformamide. The reaction mixture, under an argon stream, is cooled on a water bath and then 10 g of the 2,2,2-rifluoro-N-[3-(2,2,2-trifluoroacetylamino)-propyl]-N-(4-{trifluoroacetyl-[3-(2,2,2-trifluoroacetylamino)propyl]amino}butyl)acetamide obtained above (17 mmol), solubilized in 40 ml of dry dimethylformamide, are added dropwise. The reaction mixture is left for 1 hour at room temperature and then is again cooled on an ice bath and 3.66 g of tert-butyl bromoacetate (18.7 mmol) are added. The reaction mixture is stirred overnight at room temperature.

500 ml of ethyl acetate are then added and then the mixture is washed with three times 100 ml of a saturated sodium hydrogen carbonate solution, and three times 100 ml of a saturated sodium chloride solution. The organic phase is then dried over magnesium sulfate, filtered and concentrated to dryness. A yellow oil containing the expected impure product is thus isolated in the form of a tert-butyl ester.

This crude reaction product is diluted in 50 ml of dichloromethane and 50 ml of trifluoroacetic acid are added. The solution is stirred for 3 hours at room temperature. The reaction mixture is then evaporated to dryness and then diluted in 50 ml of dichloromethane. The product is then extracted with 3 times 150 ml of a saturated sodium hydrogen carbonate solution. The aqueous phase obtained is washed with 3 times 30 ml of dichloromethane and is then acidified by addition of concentrated hydrochloric acid. The product is then extracted with 3 times 300 ml of dichloromethane. The organic phase is dried over magnesium sulfate, filtered and concentrated to dryness. The purification is continued by chromatography on silica (elution: dichloromethane/methanol 8/2). 3.5 g of a yellow powder are thus recovered (total yield over the two steps: 32%).

¹H NMR (400 MHz, (CD₃)₂SO d6 at a temperature of 373K, δd in ppm): 1.62 (mt: 4H); from 1.80 to 2.00 (mt: 4H); 3.28 (mt: 2H); from 3.30 to 3.60 (mt: 10H); 4.01 (s: 2H); from 9.15 to 9.35 (unresolved complex: 1H).

### c—Synthesis of the Syn and Trans 4-{4-[(2-{3-[4-(3-aminopropylamino)butylamino]propylamino}acetylamino)-methyl]-[1,3]dioxolan-2-yloxy}-N,N-dioctadecyl-butyramides tetraacetate (acid-sensitive compound A)

This synthesis is performed in three steps: condensation of the two molecules whose synthesis has just been described in a and b, and then deprotection of the polyamine and finally salification. The same protocol was used for the products Syn and Trans.

### 1) Synthesis of the Syn and Trans N,N-dioctadecyl-4-(4-{[2-(trifluoroacetyl-{3-[trifluoroacetyl-(4-{trifluoroacetyl-[3-(2,2,2-trifluoroacetylamino)propyl]amino}-butyl)amino]propyl}amino)acetylamino]methyl}-[1,3]-dioxolan-2-yloxy)butyramides

800 mg of 4-(4-aminomethyl-[1,3]dioxolan-2-yloxy)-N,N-dioctadecylbutyramide (1.13 mmol Syn or Trans) obtained above (step a) dissolved in 10 ml of dichloromethane are successively supplemented with 390 μl of triethylamine (2.8 mmol), 800 mg of (trifluoroacetyl-{3-[trifluoroacetyl-(4-{trifluoroacetyl-[3-(2,2,2-trifluoroacetylamino)propyl]amino}-butyl)amino]propyl}amino)acetic acid (1.24 mmol) obtained above in step b and 600 mg of BOP. The solution is stirred for 2 hours at room temperature.

The crude reaction product is then concentrated, taken up in 150 ml of ethyl acetate, washed with three times 40 ml of a saturated sodium hydrogen carbonate solution and then three times 40 ml of a saturated sodium chloride solution. After drying over magnesium sulfate, filtration and evaporation, the product is purified by chromatography on silica (elution: ethyl acetate). 1.1 g of white powder are thus isolated (yield:73%).

### 2) Preparation of the Syn and Trans 4-{4-[(2-{3-[4-(3-aminopropylamino)butylamino]propylamino}acetylamino)-methyl]-[1,3]dioxolan-2-yloxy}-N,N-dioctadecyl-butyramides

290 mg of N,N-dioctadecyl-4-(4-{[2-(trifluoroacetyl-{3-[trifluoroacetyl-(4-{trifluoroacetyl-[3-(2,2,2-trifluoroacetylamino)propyl]amino}-butyl)amino]propyl}amino)acetylamino]methyl}-[1,3]-dioxolan-2-yloxy)butyramide (0.22 mmol, Syn or Trans) which were obtained above are dissolved in 3 ml of tetrahydrofuran, and 3 ml of molar sodium hydroxide at 4% are added with vigorous stirring. The reaction is left overnight at room temperature.

The solvent is then concentrated and then the crude product is taken up in a dichloromethane/methanol 1/1 mixture. This crude solution is purified by chromatography on silica (dichloromethane/methanol/ammonia, 45/45/10). The product is concentrated and then freeze-dried after addition of water. 180 mg of white freeze-dried product are thus obtained (yield: 87%).

### 3) Preparation of the Diastereoisomers Syn and Trans of 4-{4-[(2-{3-[4-(3-aminopropylamino)butylamino]propyl-amino}acetylamino)methyl]-[1,3]dioxolan-2-yloxy}-N,N-dioctadecylbutyramide (compound A)

The product obtained in the preceding step, in the form of a free base, is then quantitatively salified on an ion-exchange resin: it is solubilized in a water/ethanol mixture and is eluted in a column containing a large excess of acetate resin (BIO-RAD; AG 1-X2 Resin).

¹H NMR for the compound SYN (400 MHz, CDCl₃, δd in ppm): 0.89 (t, J=7 Hz: 6H); from 1.20 to 1.40 (mt: 60H); 1.52 (mt: 4H); from 1.65 to 1.90 (mt: 8H); 1.93 (mt: 2H); 1.97 (s: 3H); 2.37 (t, J=7 Hz: 2H); 2.73 (mt: 4H); 2.81 (t, J=6.5 Hz: 2H); 2.89 (mt: 4H); 2.95 (t, J=6.5 Hz: 2H); from 3.15 to 3.30 (mt: 4H); 3.32 (AB, J=17 Hz: 2H); 3.45 (dt, J=14 and 6.5 Hz: 1H); from 3.55 to 3.65 (mt: 1H); 3.61 (split t, J=7 and 2 Hz: 2H); 3.74 (t, J=8 Hz: 1H); 4.06 (t, J=8 Hz: 1H); 4.31 (mt: 1H); 5.79 (s : 1H), 7.81 (t, J=5.5 Hz: 1H).

¹H NMR for the compound TRANS (400 MHz, CDCl₃, δd in ppm): 0.88 (t, J=7 Hz: 6H); from 1.05 to 1.45 (mt: 60H); 1.51 (mt: 4H); from 1.65 to 1.90 (mt: 8H); 1.92 (mt: 2H); 1.97 (s: 3H); 2.37 (t, J=7 Hz: 2H); 2.73 (mt: 4H); 2.80 (t, J=6 Hz: 2H); 2.88 (mt: 4H); 2.96 (t, J=6 Hz: 2H); from 3.15 to 3.55 (mt: 8H); 3.57 (broad t, J=6 Hz: 2H); 3.69 (dd, J=7.5 and 5.5 Hz: 1H); 4.11 (t, J=7.5 Hz: 1H); 4.43 (mt: 1H); 5.85 (s, 1H); 7.73 (broad t, J=5.5 Hz: 1H).

### Example 4

### Synthesis of 4-{4-[(2-{3-[bis(3-aminopropyl)amino]propylamino}acetylamino)methyl]-[1,3]dioxolan-2-yloxy}-N,N-ditetradecylbutyramide tetrachlorohydride

This example describes a route of synthesis of the acid-sensitive compound B, in the form of an equimolar mixture of the two diastereoisomers Syn and Trans, of formula:

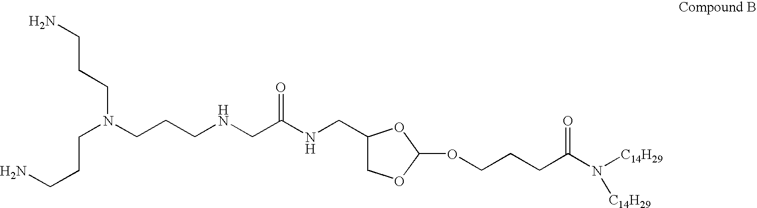

### a—Synthesis of 4-(4-aminomethyl-[1,3]dioxolan-2-yloxy)-N,N-ditetradecylbutyramide (lipid part-O-Ortho 1-NH₂)

This synthesis is performed in four steps: synthesis of the ditetradecylamine which is then functionalized to an alcohol and then attached to the group Ortho 1 whose protecting group is then cleaved.

### 1) Ditetradecylamine Hydrochloride

74 g of bromotetradecane (267.1 mmol) are supplemented with 400 ml of ethanol and 57 g of tetradecylamine (267.1 mmol). 70.8 g of sodium carbonate (667 mmol) are then placed in suspension and the reaction mixture is heated under reflux overnight. The reaction mixture is then evaporated to dryness, taken up in 1.5 l of dichloromethane and washed with 3 times 200 ml of water and then once 400 ml of a saturated sodium chloride solution. The organic phase is dried over calcium chloride and concentrated.

The salification is carried out by solubilization of the crude product in the hot state in 600 ml of isopropanol supplemented with 300 ml of 5 N hydrochloric acid in isopropanol. The clear solution thus obtained is allowed to cool, which induces crystallization of the expected product. 48.4 g of flocculant white powder are obtained after filtration and washing with isopropanol (yield: 41%).

### 2) Preparation of 4-hydroxy-N,N-ditetradecylbutyramide

22.4 g of aluminum chloride (168.1 mmol) are supplemented with 75 ml of chloroform, the whole being cooled to about 10° C. on an ice-cold water bath. 39 ml of triethylamine (280.1 mmol) in 100 ml of chloroform are added dropwise and then the reaction mixture is allowed to return to room temperature. 25 g of ditetradecylamine hydrochloride (56 mmol) mixed with 5.2 ml of butyrolactone (67.2 mmol) in 350 ml of chloroform are gradually added to the mixture, with mechanical stirring. The reaction no longer changes after 2 hours at room temperature.

200 ml of water are then added and the reaction mixture is stirred for 30 minutes. The crude product is filtered on Celite and then washed with chloroform. The filtrate is separated after settling out and the organic phase is washed with three times 150 ml of a saturated sodium chloride solution. The chloroformic solution is dried over magnesium sulfate, filtered and concentrated. 21.2 g of white powder are obtained with a yield of 76% after chromatography on silica (ethyl acetate/cyclohexane 1/1).

### 3) Preparation of N,N-ditetradecyl-4-{4-[(2,2,2-trifluoroacetylamino)methyl]-[1,3]dioxolan-2-yloxy}butyramide

3.5 g of 4-hydroxy-N,N-ditetradecylbutyramide (7.1 mmol) obtained in the preceding step are mixed with 1.8 g of 2,2,2-trifluoro-N-(2-methoxy-[1,3]dioxolan-4-ylmethyl)acetamide (Ortho 1, 7.8 mmol) and 18 mg of pyridinium para-toluenesulfonate (PPTS, 0.071 mmol). The whole is heated without solvent at 80° C. for 3 hours.

The crude reaction product is then dissolved in 200 ml of heptane, washed with 3 times 50 ml of a saturated sodium hydrogen carbonate solution, with 3 times 50 ml of acetonitrile and is then concentrated to dryness.

A small fraction of the crude product is purified on silica in order to characterize this intermediate, the remainder being used as it is for the next step. The chromatography on silica (cyclohexane/ethyl acetate 7/3 V/V) allows us to isolate a few mg of oily product.

### 4) Preparation of 4-(4-aminomethyl-[1,3]dioxolan-2-yloxy)-N,N-ditetradecylbutyramide

The crude product obtained in the preceding step is solubilized in 20 ml of tetrahydrofuran supplemented with 20 ml of sodium hydroxide at 4%. The reaction mixture is left overnight with vigorous stirring at room temperature.

The tetrahydrofuran is then concentrated and then the product is extracted with 3 times 200 ml of diethyl ether. The organic phase is dried over calcium chloride, filtered and evaporated. Chromatography on silica (dichloromethane/methanol 9/1 V/V) makes it possible to isolate 1.6 g of a colorless oil (yield on the two steps: 38%).

¹H NMR (300 MHz, CDCl₃, δd in ppm): a mixture of two diastereoisomers in the proportions 50/50 is observed.

* 0.89 (t, J=7 Hz: 6H); from 1.15 to 1.45 (mt: 44H); 1.52 (mt: 4H); 1.58 (unresolved complex: 2H); 1.95 (quintuplet, J=6.5 Hz: 2H); 2.39 (t, J=6.5 Hz: 2H); from 2.75 to 3.00 (mt: 2H); 2.31 (mt: 2H); 3.29 (mt: 2H); 3.60 (mt: 2H); 3.71 and 3.80 (respectively dd, J=7.5 Hz and 6 Hz and t, J=7.5 Hz: 1H in total); 4.06 and 4.14 (2 t, J=7.5 Hz: 1H in total); 4.21 and 4.33 (2 mts: 1H in total); 5.82 and 5.85 (2 s: 1H in total).

### b—Synthesis of [(3-{bis[3-(2,2,2-trifluoroacetylamino)propyl]amino}propyl)trifluoroacetylamino]acetic acid in the form of a trifluoroacetate salt (hydrophilic part of the polyamine-COOH type)

The proposed synthesis is performed in 6 steps starting with 3-aminopropanol and 3,3′-iminobispropylamine.

### 1) Preparation of 2,2,2-trifluoro-N-{3-[3-(2,2,2-trifluoroacetylamino)propylamino]propyl}acetamide

35 g of 3,3′-iminobispropylamine (266.7 mmol) are solubilized in 150 ml of anhydrous tetrahydrofuran under an argon stream. The reaction mixture is then cooled to 0° C. on an ice bath and 65 ml of ethyl trifluoroacetate (546.8 mmol) are added dropwise (very slowly) using a dropping funnel. At the end of the addition (3 hours later), the reaction mixture is allowed to return to room temperature and the stirring is maintained for a few hours under argon.

The reaction mixture is then filtered on paper. The filtrate is concentrated to dryness, taken up several times in dichloromethane, the final drying being carried out in an oven at 40° C. under the vacuum produced by a slide vane rotary vacuum pump for 16 hours. 85.3 g of a white powder are isolated pure without further purification (yield: 99%).

### 2) Preparation of tert-butyl (3-hydroxypropylamino)acetate

196 ml of 3-aminopropanol (2.56 mol) are diluted in 250 ml of dichloromethane and the whole is cooled to 0° C. on an ice bath. 20 g of tert-butyl bromoacetate (102.5 mmol), solubilized in 200 ml of dichloromethane, are then added dropwise while the reaction mixture is maintained at 0° C. At the end of the addition (2 hours later), the reaction mixture is left at room temperature for 3 hours.

This crude product is then washed with 3 times 150 ml of a saturated sodium hydrogen carbonate solution, and then 3 times 150 ml of a saturated sodium chloride solution. The organic phase is dried over calcium chloride, filtered and concentrated. 17.8 g of a colorless oil are thus isolated (yield: 92%).

### 3) Preparation of tert-Butyl [(3-hydroxypropyl)-trifluoroacetylamino]acetate

17.65 g of tert-butyl (3-hydroxypropylamino)acetate (93.3 mmol) are solubilized in 100 ml of dichloromethane and the whole is cooled to 0° C. on an ice bath. 26 ml of triethylamine (186.6 mmol) are added, followed by a dropwise addition of 21.5 g of trifluoroacetic anhydride (102.6 mmol) using a dropping funnel. At the end of the addition, the reaction mixture is left at room temperature overnight, with magnetic stirring.

This solution is then washed with 3 times 50 ml of a saturated sodium hydrogen carbonate solution, 3 times 50 ml of a 0.5 M potassium hydrogen sulfate solution, and then 3 times 50 ml of a saturated sodium chloride solution. The organic phase is dried over magnesium sulfate, filtered and concentrated. 24.5 g of a pale yellow oil are thus isolated (yield: 92%).

### 4) Preparation of tert-butyl [(3-bromopropyl)trifluoroacetylamino]acetate

10 g of tert-butyl [(3-hydroxypropyl)-trifluoroacetylamino]acetate (35 mmol) are solubilized in 150 ml of tetrahydrofuran. 12.4 g of triphenylphosphine (47.3 mmol) are added and the reaction mixture is thermostated at 15-20° C. 15.1 g of carbon tetrabromide (45.6 mmol), dissolved in 60 ml of acetonitrile, are added dropwise using a dropping funnel and the whole is stirred at room temperature for 4 hours.

The reaction mixture is then concentrated to dryness, taken up in ethyl acetate and filtered on paper. The filtrate is concentrated to dryness, taken up in cyclohexane and filtered on sintered material No. 3. The filtrate is again concentrated and purified by chromatography on silica (cyclohexane/ethyl acetate 8/2 V/V). 10.4 g of a pale yellow oil are thus isolated (yield: 85%).

### 5) Preparation of tert-butyl [(3-{bis[3-(2,2,2-trifluoroacetylamino)propyl]amino}propyl)trifluoroacetylamino]acetate

26 g of tert-butyl [(3-bromopropyl)trifluoroacetylamino]acetate (74.7 mmol) and 24.1 g of 2,2,2-trifluoro-N-{3-[3-(2,2,2-trifluoroacetylamino)propylamino]propyl}acetamide (74.7 mmol) are solubilized in 130 ml of acetonitrile. 30 g of potassium carbonate (224 mmol) are then placed in suspension and the whole is heated under reflux for 6 hours.

The reaction mixture is then filtered on paper and concentrated to dryness. The crude product is then purified by chromatography on silica (cyclohexane/ethyl acetate 2/8 V/V). 16.6 g of a pale yellow oil (yield: 38%) are thus isolated.

### 6) Preparation of the trifluoroacetate salt of [(3-{bis[3-(2,2,2-trifluoroacetylamino)propyl]amino}-propyl)trifluoroacetylamino]acetic acid

15.8 g of tert-butyl [(3-{bis[3-(2,2,2-trifluoroacetylamino)propyl]amino}propyl)trifluoroacetylamino]acetate (28.76 mmol) are supplemented with 50 ml of dichloromethane and then with 50 ml of trifluoroacetic acid. This mixture is stirred for a few hours at room temperature. The reaction mixture is then concentrated to dryness. 18.7 g of pale yellow honey are thus isolated (yield: 100%).

¹H NMR (300 MHz, CDCl₃, δd in ppm): at room temperature, a mixture of rotamers is observed. * from 1.80 to 2.10 (mt: 6H); 3.12 (mt: 6H); 3.29 (mt: 4H); 3.50 (mt: 2H); 4.13 and 4.29 (respectively broad s and mt: 2H in total); from 9.50 to 9.75 (mt: 2H)

### c—Synthesis of 4-{4-[(2-{3-[bis(3-aminopropyl)amino]-propylamino}acetylamino)methyl]-[1,3]dioxolan-2-yloxy}-N,N-ditetradecylbutyramide tetrahydrochloride (compound B)

This synthesis is performed in three steps: condensation of the two molecules obtained in parts a and b above, and then deprotection of the polyamine and salification.

### 1) Preparation of 4-[4-({2-[(3-{bis[3-(2,2,2-trifluoroacetylamino)propyl]amino}propyl)trifluoroacetylamino]-acetylamino}methyl)-[1,3]dioxolan-2-yloxy]-N,N-ditetradecylbutyramide

1.9 ml of triethylamine (13.8 mmol), 2 g of [(3-{bis[3-(2,2,2-trifluoroacetylamino)propyl]amino}-propyl)trifluoroacetylamino]acetic acid (trifluoroacetate salt, 3.04 mmol) and 1.8 g of BOP (4.14 mmol) are successively added to 1.65 g of 4-(4-aminomethyl-[1,3]dioxolan-2-yloxy)-N,N-ditetradecylbutyramide (2.76 mmol) dissolved in 30 ml of dichloromethane. The solution is stirred for 1 hour at room temperature.

The crude reaction product is then concentrated to dryness, taken up in 200 ml of ethyl acetate, washed with 40 ml of a saturated sodium chloride solution, and then 3 times 40 ml of a saturated sodium hydrogen carbonate solution, and then 3 times 40 ml of a saturated sodium chloride solution. The product is then purified by chromatography on silica (elution: ethyl acetate). 2.2 g of pale yellow honey are thus isolated (yield: 72%).

### 2) Preparation of 4-{4-[(2-{3-[bis(3-aminopropyl)-amino]propylamino}acetylamino)methyl]-[1,3]dioxolan-2-yloxy}-N,N-ditetradecylbutyramide

2.1 g of 4-[4-({2-[(3-{bis[3-(2,2,2-trifluoroacetylamino)propyl]amino}propyl)trifluoroacetylamino]acetylamino}methyl)-[1,3]dioxolan-2-yloxy]-N,N-ditetradecylbutyramide (1.88 mmol) are dissolved in 30 ml of tetrahydrofuran, and 30 ml of molar sodium hydroxide at 4% are added, with vigorous stirring. The reaction is left overnight at room temperature.

The solvent is then concentrated and then the crude product is taken up in a dichloromethane/methanol 1/1 mixture. This crude solution is purified by chromatography on silica (dichloromethane/methanol/ammonia, 45/45/10, V/V/V). The product is concentrated and then freeze-dried after addition of water. 1.3 g of white freeze-dried product are thus obtained (yield: 84%).

### 3) Preparation of 4-{4-[(2-{3-[bis(3-aminopropyl)amino]propylamino}acetylamino)methyl]-[1,3]dioxolan-2-yloxy}-N,N-ditetradecylbutyramide tetrahydrochloride (compound B)

The product obtained in the preceding step in the form of a free base is then quantitatively salified on an ion-exchange resin: it is solubilized in water, and eluted in a column containing a large excess of chloride resin (FLUKA; DOWEX 21K). The structure of the white freeze-dried product obtained is confirmed by ¹H NMR.

¹H NMR (300 MHz, (CD₃)₂SO d6, δd in ppm): a mixture of two diastereoisomers in the proportions 50/50 is observed.

* 0.89 (t, J=7 Hz: 6H); from 1.15 to 1.40 (mt: 44H); from 1.40 to 1.60 (mt: 4H); 1.72 (mt: 8H); 2.31 (mt: 2H); from 2.40 to 2.55 (mt: 6H); from 2.70 to 2.90 (mt: 6H); from 3.15 to 3.75-from 4.00 to 4.40 (2 series of mt: 13H in total); 5.83 and 5.86 (2 s: 1H in total).

### Example 5

### Synthesis of the PEGoylated Lipids C and D

This example describes a route of synthesis of the pegoylated lipids Octadecanol-Ortho 1-PEG₅₀₀₀-OMe and Cholesterol-Ortho 1-PEG₅₀₀₀-OMe which differ from each other only in their lipid portion: octadecanol for compound C and cholesterol for compound D. These two acid-sensitive compounds have the general formula:

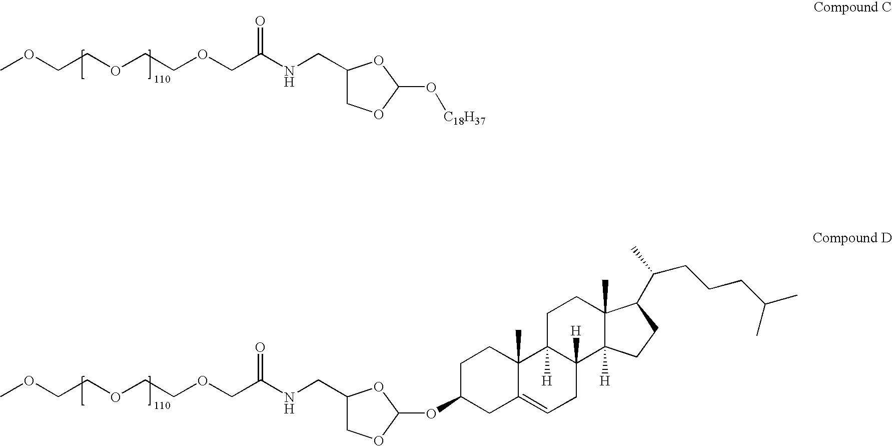

### a—Synthesis of C-(2-octadecyloxy-[1,3]dioxolan-4-yl)methylamine and of C-{2-[17-(1,5-dimethylhexyl)-10,13-dimethyl-2,3,4,7,8,9,10,11,12,13,14,15,16,17-tetradecahydro-1H-cyclopenta[a]phenanthren-3-yloxy]-[1,3]dioxolan-4-yl}methylamine (lipid parts-O-Ortho 1-NH₂)

This synthesis is performed in two steps starting with the compound Ortho 1 by substitution of the exocyclic methoxy group with the fatty alcohol (cholesterol or octadecanol) and then deprotection of the amine.

### 1) Preparation of 2,2,2-trifluoro-N-(2-octadecyloxy-[1,3]dioxolan-4-ylmethyl)acetamide

3 g of 2,2,2-trifluoro-N-(2-methoxy-[1,3]dioxolan-4-ylmethyl)acetamide (Ortho 1, 13.09 mmol) are mixed with 3.54 g of octadecanol (13.09 mmol). The mixture is melted at 80° C. and left for 2 hours after the addition of 32 mg of pyridinium para-toluenesulfonate (0.13 mmol). The crude reaction product is then dissolved in cyclohexane, washed with a saturated sodium hydrogen carbonate solution and then with a saturated sodium chloride solution, dried over magnesium sulfate and then concentrated to dryness. This crude product is used as it is for the next step.

### 1′) Preparation of N-{2-[17-(1,5-dimethylhexyl)-10,13-dimethyl-2,3,4,7,8,9,10,11,12,13,14,15,16,17-tetradecahydro-1H-cyclopenta[a]phenanthren-3-yloxy]-[1,3]dioxolan-4-ylmethyl)-2,2,2-trifluoroacetamide

The protocol is identical to that described above, it being possible for the reaction to also be performed without catalyst.

### 2) Preparation of C-(2-octadecyloxy-[1,3]dioxolan-4-yl)methylamine

6.12 g of crude reaction product of the preceding step 1 (13.09 mmol) are dissolved in 20 ml of tetrahydrofuran. The reaction mixture is cooled on an ice bath, and 30 ml of sodium hydroxide at 4% are added. The mixture is stirred at room temperature until complete disappearance of the reagent is obtained (over a period of 4 hours).

Next, the solvent is concentrated in part and then-extracted with 3 times 200 ml of diethyl ether. The organic phase is dried over calcium chloride, filtered and evaporated. The crude reaction product is purified by chromatography on silica (dichloromethane/methanol 9/1, V/V). 2.6 g of a white powder are thus recovered (yield: 53% on the two consecutive steps 1 and 2).

¹H NMR (300 MHz, CDCl₃, δd in ppm). A mixture of two diastereoisomers in the approximate proportions 50/50 is observed.

* 0.89 (t, J=7 Hz: 3H); from 1.20 to 1.45 and 1.58 (2 mts: 32H in total); from 2.75 to 3.00 (mt: 2H); 3.53 (mt: 2H); from 3.65 to 3.85 (mt: 1H); from 4.00 to 4.40 (mt: 2H); 5.81 and 5.84 (2 s: 1H in total).

### 2′) Preparation of C-{2-[17-(1,5-dimethylhexyl)-10,13-dimethyl-2,3,4,7,8,9,10,11,12,13,14,15,16,17-tetradecahydro-1H-cyclopenta[a]phenanthren-3-yloxy]-[1,3]dioxolan-4-yl}methylamine

The protocol is identical to that of step 2). 1.4 g of white powder are thus recovered (yield: 22% on the two consecutive steps 1′ and 2″).

¹H NMR (400 MHz, CDCl₃, δd in ppm). A mixture of two diastereoisomers in the approximate proportions 50/50 is observed.

* 0.69 (s: 3H); from 0.85 to 1.75-1.86 and 2.00 (mts: 26H in total); 0.88 (mt: 6H); 0.93 (d, J=7 Hz: 3H); 1.01 (s: 3H); 2.33 (mt: 2H); from 2.75 to 3.00 (mts: 2H); 3.50 (mt: 1H); 3.71-3.85-4.05 and 4.16 (4 mts: 2H in total); 4.20 and 4.35 (2 mts: 1H in total); 5.36 (mt: 1H); 5.93 and 5.96 (2 s: 1H in total).

### b—Synthesis of the acid of methoxypolyethylene glycol 5000 (hydrophilic part MeO-PEG₅₀₀₀-COOH)

A single step is necessary: oxidation of the terminal hydroxyl group of the commercial methoxypolyethylene glycol.

20 g of MeO-PEG₅₀₀₀-OH (4 mmol) are dissolved in 100 ml of an equal volume water/acetonitrile mixture. 312 mg of 2,2,6,6-tetramethylpiperidinyloxyl (2 mmol) and then 6.4 g of [bis(acetoxy)iodo]benzene (20 mmol) are added and the reaction mixture is left stirring for 16 hours at room temperature.

This crude reaction product is then evaporated to dryness, taken up in 40 ml of a dichloromethane/ethanol (1/1; V/V) mixture, and then precipitated by addition of 500 ml of diethyl ether. 19 g of a white powder are thus isolated by filtration and washing with ether (yield: 95%).

¹H NMR (300 MHz, CDCl₃, δd in ppm): 3.39 (s: 3H); from 3.40 to 3.95 (mts: 404H); 4.15 (s: 2H).

### c—Synthesis of methoxy-(polyethylene glycol 5000)-(N-(2-octadecyloxy-[1,3]dioxolan-4-ylmethyl)amide (compound C) and of methoxy-(polyethylene glycol 5000)-N-{2-[17-(1,5-dimethylhexyl)-10,13-dimethyl-2,3,4,7,8,9,10,11,12,13,14,15,16,17-tetradecahydro-1H-cyclopenta[a]-phenanthren-3-yloxy]-[1,3]dioxolan-4-ylmethyl}amide (compound D)

Compound C:

1.2 g of MeO-PEG₅₀₀₀-COOH (0.24 mmol) obtained in the preceding steps are dissolved in 5 ml of dichloromethane. 188 μl of triethylamine (1.34 mmol) are added and then 100 mg of C-(2-octadecyloxy-[1,3]dioxolan-4-yl)methylamine (0.27 mmol). 143 mg of BOP (0.32 mmol, 1.2 eq) are then added and the reaction is left stirring at room temperature for one hour.

The reaction mixture is precipitated by addition of diethyl ether (60 ml), centrifuged, washed with ether and then injected into preparative high-performance liquid chromatography (HPLC). By isolating the purest fractions, 415 mg of white freeze-dried product are thus obtained (yield: 32%).

¹H NMR (300 MHz, CDCl₃, δd in ppm): a mixture of two diastereoisomers in the approximate proportions 50/50 is observed.

* 0.90 (t, J=7 Hz: 3H); from 1.20 to 1.40 and from 1.50 to 1.75 (mts: 32H); 3.39 (s: 3H); from 3.40 to 3.95 (mts: 448H); 4.02 and 4.03 (2s: 2H in total); from 4.00 to 4.25 (mts: 3H in total); 5.80 and 5.84 (2s: 1H in total).

Compound D:

The protocol is identical to that presented for the preparation of compound C. By isolating the purest fractions, 395 mg of white freeze-dried product are thus obtained (yield: 30%).

¹H NMR (400 MHz, CDCl₃, δd in ppm): a mixture of two diastereoisomers in the approximate proportions 50/50 is observed.

* 0.68 (s: 3H); from 0.85 to 1.75-1.85 and 2.00 (mts: 26H in total); 0.87 (mt: 6H); 0.92 (d, J=7 Hz: 3H); 1.00 (s: 3H); 2.33 (mt: 2H); 3.39 (s: 3H); from 3.40 to 3.90 (mts: 448H); from 4.00 to 4.20 (mts: 1H in total); 4.01 and 4.04 (2s: 2H in total); 4.28 and 4.46 (2 mts: 1H in total); 5.35 (mt: 1H); 5.90 and 5.95 (2s: 1H in total).

### Example 6

### Synthesis of the PEGoylated lipid E

This example describes a route of synthesis of the pegoylated lipid octadecanol-Ortho 2-PEG₅₀₀₀-OMe which has the general formula:

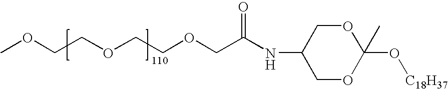

### a—Synthesis of 2-methyl-2-octadecyloxy-[1,3]dioxan-5-ylamine (hydrophobic lipid part-O-Ortho2-NH₂)

This synthesis if performed in two steps starting with the substrate Ortho 2 whose synthesis is described above, by substitution of the exocyclic methoxy group with octadecanol and then deprotection of the amine.

### 1) Preparation of 2,2,2-trifluoro-N-(2-methyl-2-octadecyloxy-[1,3]dioxan-5-yl)acetamide

3 g of 2,2,2-trifluoro-N-(2-methoxy-2-methyl-[1,3]dioxan-5-yl)acetamide (12.34 mmol) are mixed with 3 g of octadecanol (11.1 mmol). The mixture is melted at 80° C. and left for 2 hours in order to evaporate all the methanol produced during the alcohol exchange.

The molten reaction mixture is then poured into 50 ml of acetonitrile, which induces its precipitation. The expected product is purified by recrystallization from acetonitrile. 2.85 g of white powder are thus isolated (yield: 53% after recrystallization).

### 2) Preparation of 2-methyl-2-octadecyloxy-[1,3]dioxan-5-ylamine

1 g of the trifluoroacetamide derivative of the preceding step (2.08 mmol) is dissolved in 10 ml of tetrahydrofuran, to which 10 ml of molar sodium hydroxide at 4% are added. The mixture is vigorously stirred at room temperature until complete disappearance of the reagent is obtained (over a period of 4 hours).

Next, the solvent is concentrated in part and then extracted with 3 times 100 ml of diethyl ether. The organic phase is dried over calcium chloride, filtered and evaporated to dryness. 810 mg of white powder are thus isolated without purification (yield: 100%).

¹H NMR (400 MHz, CDCl₃, δd in ppm): 0.89 (t, J=7 Hz: 3H); from 1.20 to 1.50 (mt: 30H); 1.49 (s: 3H); 1.62 (mt: 2H); 1.67 (broad s: 2H); 2.71 (mt: 1H); 3.46 (t, J=7 Hz: 2H); 3.54 (broad d, J=10 Hz: 2H); 4.30 (dd, J=10 and 1.5 Hz: 2H).

### b—Synthesis of methoxy-(polyethylene glycol 5000)-N-(2-methyl-2-octadecyloxy-[1,3]dioxan-5-yl)amide (Compound E)

The last step consists in condensing the lipid amine with the acid of PEG (whose synthesis is described in example 5).

1.15 g of MeO-PEG₅₀₀₀-COOH (0.23 mmol) are dissolved in 5 ml of dichloromethane. 181 μl of triethylamine (1.30 mmol) are added and then 100 mg of 2-methyl-2-octadecyloxy-[1,3]dioxan-5-ylamine (0.26 mmol). 172 mg of BOP (0.39 mmol) are then added and the reaction is left stirring at room temperature for one hour.

The reaction mixture is precipitated by addition of diethyl ether (60 ml), centrifuged, washed with ether and then injected into preparative HPLC. By isolating the purest fractions, 420 mg of white freeze-dried product are thus obtained (yield: 34%).

¹H NMR (400 MHz, CDCl₃, δd in ppm); 0.89 (t, J=7 Hz: 3H); from 1.20 to 1.50 (mt: 30H); 1.48 (s: 3H); from 1.55 to 1.75 (mt: 2H); 3.39 (s: 3H); from 3.40 to 3.90 (mt: 448H); 3.89 (broad d, J=8.5 Hz: 1H); 4.05 (s: 2H); 4.30 (broad d, J=12 Hz: 2H); 7.57 (d, J=8.5 Hz: 1H).

### Example 7

### Study of the Compaction of DNA by the Acid-Sensitive Compounds A Syn and Trans

The acid-sensitive compounds A forms Syn and Trans prepared above have a structure analogous to the cationic lipids conventionally used for the nonviral transfection of DNA, and they possess, inter alia, in their structure a cyclic ortho-ester function which contributes to making them acid-sensitive.

The aim of this example is therefore to demonstrate that the acid-sensitive compounds A Syn and Trans preserve the power to compact DNA to be transfected specific to the cationic lipids, while having the capacity to become degraded in acidic medium and therefore to release the compacted DNA. This can be easily shown by a fluorescence test with ethidium bromide (EtBr): the absence of fluorescence reflects the absence of free DNA, which means that the DNA is compacted.

In the text which follows, the two forms Syn and Trans of the acid-sensitive compound A as prepared in example 3 were used and the non-acid-sensitive analog described in the publication WO 97/18185, and which has the formula:
H₂N—(CH₂)₃—NH—(CH₂)₄—NH—(CH₂)₃—NH—CH₂—CO-Gly-N[(CH₂)₁₇—CH₃]₂
was used as control. This non-acid-sensitive analog is called in the text which follows “control cationic lipid”.

The DNA is brought into contact with increasing quantities of control cationic lipid or of acid-sensitive compound A Syn or Trans, by mixing an equal volume of lipid solutions of different titers with the DNA solutions. Samples of 800 μl of DNA complexes having a concentration of 10 μg/ml are thus prepared in a sodium chloride solution at 150 mM with increasing quantities of control cationic lipid or of acid-sensitive compound A Syn or Trans.

At time t=0, 200 μl of buffer having a concentration of 0.1 mol/l at pH 5 are added to these samples and the samples are stored in an oven at 37° C. The fluorescence with ethidium bromide (EtBr) is measured over time (measurement at 20° C.) using a FluoroMax-2 (Jobin Yvon-Spex), with excitation and emission wavelengths of 260 nm and 590 nm respectively. The slit widths for excitation and for emission are set at 5 nm. The fluorescence value is recorded after addition of 3 μl of ethidium bromide at 1 g/l per ml of DNA/cationic lipid or DNA/acid-sensitive compound solution (at 0.01 mg of DNA/ml).

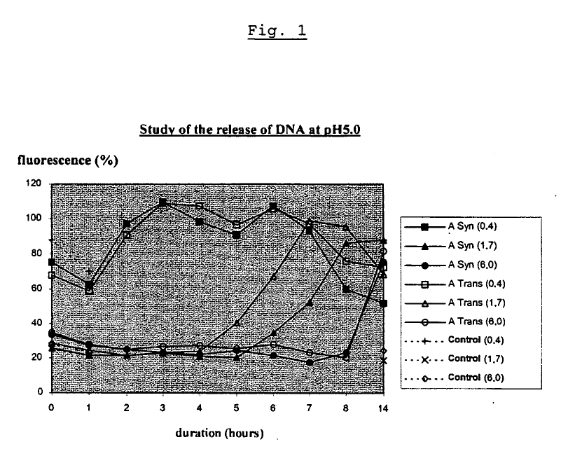

The results are summarized in FIG. 1. At pH 5, when the quantity of acid-sensitive compound A Syn or Trans or of control cationic lipid used to compact the DNA is too low (0.4 nmol lipid/μg of DNA) and when the DNA is therefore not completely compacted, no significant variation in fluorescence is measurable over time. On the other hand, a different behavior of the acid-sensitive compounds A Syn and Trans is observed with respect to the (non-acid-sensitive) control cationic lipid for larger quantities (1.7 nmol lipid/μg of DNA and 6.0 nmol lipid/μg of DNA) which allow complete compaction of the DNA. Indeed, the acid-sensitive compounds A Syn and Trans release the DNA over time as demonstrated by the increase in fluorescence, which is not the case with the control cationic lipid which is not acid-sensitive. It is observed, in addition, that this release of DNA occurs a few hours after the addition of acid (at pH 5 and 37° C.).

A shift is also observed in the kinetics of release of the DNA according to the quantity of acid-sensitive compound used: the lower the quantity of acid-sensitive compound A used, the more rapid the release of the DNA.

This study demonstrates the remarkable properties of the acid-sensitive compounds A Syn and Trans: they are capable of forming complexes with DNA by compacting it, and their degradation in acidic medium causes a degradation of the complexes formed with the DNA, and therefore the release of the DNA. These acid-sensitive compounds are therefore particularly useful in the context of the nonviral transfection of DNA into cells.

### Example 8

### Study of the Transfecting Power of the Acid-Sensitive Compounds A Syn and Trans in Vitro

This example illustrates the in vitro transfecting power of the acid-sensitive compounds A Syn and Trans, compared with their non-acid-sensitive analog described in the preceding example (the control cationic lipid).

This study was carried out at 4 different charge ratios: 1.0 or 4.0 or 6.0 or 10.0 nmol of lipid/μg of DNA. Each of these conditions was tested with and without fetal calf serum (“+ or − Serum”)

Culture of the cells: HeLa cells (American type Culture Collection (ATCC) Rockville, Md., USA) derived from a human cervical epithelium carcinoma, are cultured in the presence of a medium of the MEM (“minimum essential medium”) type with addition of 2 mM L-glutamine, 50 units/ml of penicillin, and 50 units/ml of streptomycin. The medium and the additives are obtained from Gibco/BRL life Technologies (Gaithersburg, Md., USA). The cells are cultured in flasks at 37° C. and at 5% carbon dioxide in an incubator.

Transfection: a day before the transfection, the HeLa cells are transferred into 24-well plates with a cell number of 30,000 to 50,000 per well. These dilutions represent approximately 80% confluence after 24 hours.

For the transfection, the cells are washed twice and incubated at 37° C. with 500 μl of medium with serum (10% FCS v/v) or without serum.

50 μl of complexes containing 0.5 μg of plasmid DNA are added to each well (the complexes are prepared at least 30 minutes before the addition to the well). After two hours at 37° C., the plates without serum are supplemented with 10% (v/v) of FCS (“Fetal Calf Serum”).

All the plates are placed for 36 hours at 37° C. and at 5% carbon dioxide.

Determination of the luciferase activity: Briefly, the transfected cells are washed twice with 500 μl of PBS (phosphate buffer) and then lysed with 250 μl of reagent (Promega cell culture lysis reagent, from the Luciferase Assay System kit).

An aliquot of 10 μl of supernatant of the lysate centrifuged (12,000×g) for 5 minutes at 4° C. is measured with the Wallac Victor2 luminometer (1420 Multilabel couter).

The luciferase activity is assayed by the emission of light in the presence of luciferin, of coenzyme A and of ATP for 10 seconds and expressed relative to 2000 treated cells. The luciferase activity is thus expressed as Relative Light Unit (“RLU”: “Relative light unit”) and normalized with the concentration of proteins in the sample obtained using a Pierce BCA kit (Rockford, Ill., USA).

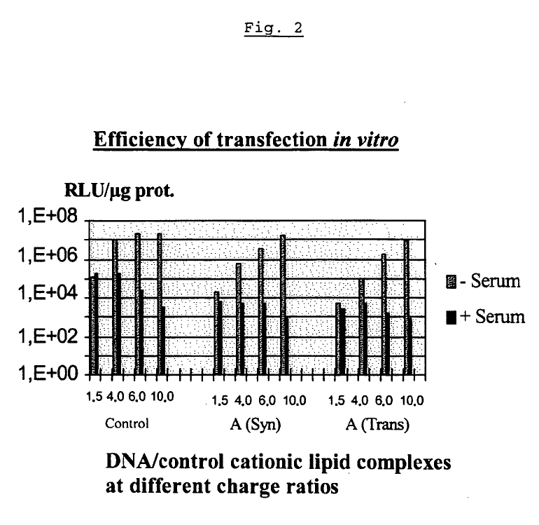

The results, summarized in FIG. 2, show a high transfection activity for the three compounds tested (the acid-sensitive compounds A Syn and Trans and the control cationic lipid). No significant difference is observed between them. In the absence of serum, the level of transfection is high in all the cases (10⁵ to 10⁷ RLU/μg of protein) and the transfecting power increases with the quantity of acid-sensitive compound or of control cationic lipid used. The presence of serum induces inhibition of transfection in all cases.

This example therefore shows that the transfecting power of the acid-sensitive compound A in its Syn and Trans forms is preserved compared with its non-acid-sensitive analog (the control cationic lipid). More generally, the introduction of an acid-sensitive cyclic ortho-ester function into molecules of the cationic lipid type which are known to be useful in nonviral transfection does not destroy the capacity of these compounds to efficiently transfect DNA.

### Example 9

### Use of the Acid-Sensitive Compounds C and D as Nonionic Surfactants for the Colloidal Stabilization of DNA/cationic Lipid Transfecting Complexes

This example illustrates the fact that the acid-sensitive pegoylated lipids of the type defined under (d) in the present application can be used as nonionic surfactants which play a colloidal stabilizing role with respect to the DNA/cationic lipid transfecting particles.

In the present example, the cationic lipid used is that already used in examples 7 and 8 and described in the publication WO 97/18185 under the formula:
H₂N—(CH₂)₃—NH—(CH₂)₄—NH—(CH₂)₃—NH—CH₂—CO-Gly-N[(CH₂)₁₇—CH₃]₂
(control cationic lipid).

Compounds C and D prepared in example 5 are used as acid-sensitive pegoylated lipids. As controls, there are used BRIJ 700 (SIGMA) and the pegoylated lipid of formula: 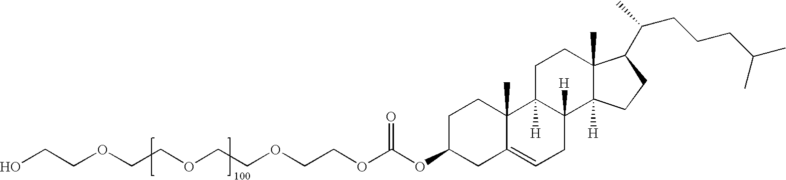
which are non-acid-sensitive analogs of compounds C and D, respectively, and which are known as nonionic surfactants (see for example the publication WO 98/34648). The two controls are used at 10 g/l in water.

1 ml samples of nucleolipid complexes (DNA/control cationic lipid) are prepared from DNA at 10 μg/ml in 75 mM of a sodium chloride solution, by mixing in equal volume the solution containing the control cationic lipid and one of the pegoylated lipids (compound C or compound D or Brij 700 or the analog D) with the DNA solution. All these samples have a control cationic lipid/DNA ratio of 1.5 (in nmol of lipid per μg of DNA) and contain increasing quantities of pegoylated lipid (expressed as polymer/DNA weight/weight ratio).

The measurement of the size of the particles obtained is made 30 minutes after the mixing and makes it possible in particular to study the influence of the quantity of acid-sensitive pegoylated lipid (compound C or D) or of the non-acid-sensitive pegoylated lipid (Brij 700 or analog D) on the stabilization of the control cationic lipid/DNA complexes. The measurement of the hydrodynamic diameter is made with a Coulter N4Plus apparatus using plastic cuvettes (four transparent sides) filled with 800 μl of the different solutions containing 0.01 mg of DNA/ml, the measurement being carried out at 90° in unimodal mode.

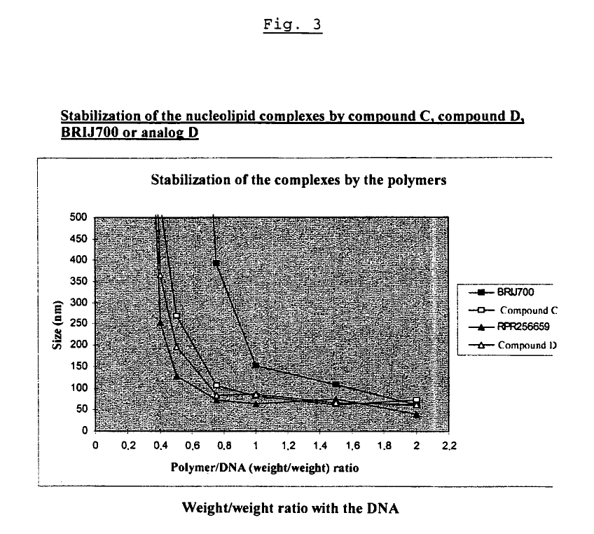

The results are presented in FIG. 3 which describes the variation in the size of the control cationic lipid/DNA particles as a function of the quantity of compound C or D or of Brij 700 or of analog D used.

It is observed that the acid-sensitive pegoylated lipids, as well as their stable controls, allow the formation of small-sized (less than 100 nm) control cationic lipid/DNA particles when a minimal quantity is reached, whereas these same control cationic lipid/DNA particles spontaneously aggregate (size greater than 1 μm) in the absence of acid-sensitive or non-acid-sensitive pegoylated lipid or when the quantity of the latter is too low.

This example thus demonstrates that compounds C and D, and more generally acid-sensitive pegoylated lipids of the type defined under (d) in the general formula (I) of the present application, can be used as nonionic surfactants, and their colloidal stabilizing power is comparable to that of the stable nonionic surfactants (that is to say non-acid-sensitive such as Brij 700 for example) which are conventionally used for the colloidal stabilization of nucleolipid particles.

### Example 10

### Influence of the pH on the Colloidal Stabilization of DNA/Cationic Vector Complexes by Compounds C and D

This example illustrates the fact that acid-sensitive pegoylated lipids of the type defined under (d) in the general formula (I) of the present application, and which are used as nonionic surfactants for stabilizing DNA/cationic lipid nucleolipid complexes, can be degraded at acidic pH and thus release said nucleolipid complexes.

The remarkable property which is used here is the absence of colloidal stabilization when a pegoylated lipid is replaced with a PEG without a lipid portion. Indeed, the formulation of the DNA in the presence of a cationic vector and of an acid-sensitive pegoylated lipid leads, in a non-buffered medium, to small particles (see example 9 above). The same study with a PEG alone (that is to say not coupled to a lipid portion) does not, on the other hand, give any stabilization.

The acid-sensitive pegoylated lipid used in this example is the compound D prepared in example 5, as well as its non-acid-sensitive analog called “Analog D” in the preceding example and in the text which follows.

1 ml samples of nucleolipid complexes (DNA/control cationic lipid) are prepared from DNA at 10 μg/ml in 75 mM of a sodium chloride solution, by mixing in equal volume the solution containing the control cationic lipid and compound D or the non-acid-sensitive analog D with the DNA solution. All these samples have a control cationic lipid/DNA ratio of 1.5 (in nmol of lipid per μg of DNA) and contain increasing quantities of pegoylated lipid (expressed as polymer/DNA weight/weight ratio).

100 μl of acetic acid/sodium acetate buffer at pH 5 (0.1 mol/l) are added to these samples thermostated at 37° C. in a ventilated oven. The size of the particles is measured as a function of time.

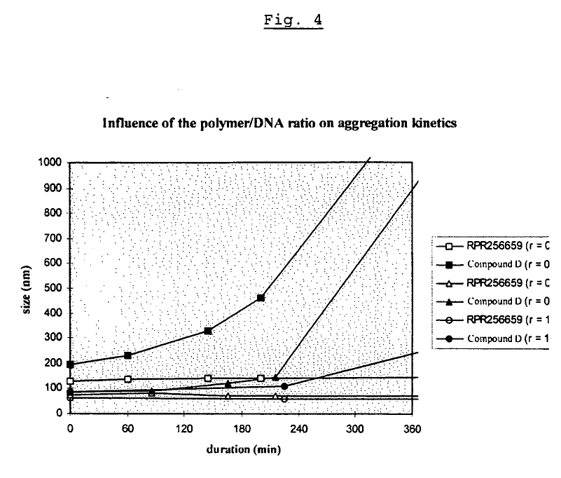

The results obtained are represented in FIG. 4 which represents the size of the nucleolipid particles as a function of time, and also according to the acid-sensitive or non-acid-sensitive pegoylated lipid/DNA ratio (3 weight/weight ratios tested: 0.5 or 0.75 or 1)

In the case of analog D (non-acid-sensitive pegoylated lipid), the pH has no influence on the colloidal stability of the particles: the particles formed have a small size, of the order of 100 nm, regardless of the analog D/DNA ratio used. At time t=0, the same stability is observed with compound D, regardless of the compound D/DNA ratio.

On the other hand, an increase in the size of the nucleolipid particles as a function of time is observed when compound D is used as nonionic surfactant at acidic pH (pH 5). The lower the compound D/DNA ratio, the more rapid this increase in size of the nucleolipid particles. Thus, after 4 hours, the particles have completely formed into aggregates in the case of a low ratio (of 0.5) and not at all or only slightly when a large excess of compound D is used (ratio of 1).

It is thus possible to deduce therefrom that compound D is sensitive to the pH value. Indeed, the increase in the size of the nucleolipid particles as a function of time reflects the degradation of the acid-sensitive pegoylated lipid (compound D) when an acidic medium is used (there is in fact “ungrafting” of the lipid portion at the level of the acid-sensitive portion of the compound).

In addition, by increasing the acid-sensitive pegoylated lipid/DNA ratio, the time necessary for the aggregation is also increased, which tends to show that the more the acid-sensitive pegoylated lipid is used, the more of it there is to be degraded before crossing the threshold beyond which aggregation of the nucleolipid particles occurs. It is thus possible, by adjusting the quantity of acid-sensitive colloidal stabilizer, to program the time necessary for the release of the active ingredient at a given pH.

### Example 11

### Transfection in Vivo of DNA Formulated with a Cationic Lipid/Neutral Co-Lipid/Compound D Mixture

This example illustrates the impact of a nonionic surfactant such as compound D on the efficiency of DNA transfer formulated with a cationic lipid-based liposome.

1) Preparation of the Liposomes

The cationic lipid of formula: 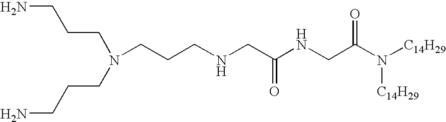
and the neutral lipid DOPE (obtained from Avanti Polar Lipids, Birmingham, Ala.) where it is dissolved in chloroform and then mixed in equimolar quantities.

Appropriate quantities of compound D were dissolved in chloroform and added to the mixture. The quantities of compound D used are given in mol % of the total quantity of lipid.

The organic solvent is then evaporated under an argon stream so that a thin lipid film forms at the bottom of the tube. This film is dried under vacuum for at least 1 hour and then rehydrated with a 20 mM HEPES buffer at pH 7.4 and 5% dextrose, at 4° C. for 2 hours. The lipid suspension thus obtained is heated at 50° C. for 30 minutes, and then sonicated for 5 minutes so as to form a homogeneous suspension of liposomes of about 100 nm.

The DNA used is plasmid DNA containing the CAT gene under the control of a CMV promoter. The plasmid DNA used possesses the following criteria: endotoxin level of less than 20 U/mg, quantity of supercoiled DNA greater than 90%, contamination with E. coli DNA less than 5%, contamination with RNA less than 5% and contamination with proteins less than 1%.

2) Preparation of the DNA/Lipid Complexes

Equivalent volumes of liposome suspension (in appropriate concentration) are rapidly added to a DNA solution while mixing rapidly and vigorously. Overall, 1 μg of DNA forms a complex with 5 nmol of cationic lipid. The complexes formed have a diameter of approximately 100 to 240 nm.

3) Administration in Vivo

About 6 week old female Balb/C mice are used in all the experiments. Each mouse receives a subcutaneous injection with 10⁶ M109 cells in the right flank. When the tumours reach a size of about 300 mm³, the mice receive 200 μl of DNA/lipid complexes containing 50 μg of DNA, by intravenous injection. The tissues are removed 24 hours after injection and stored at −70° C. until used.

4) CAT Assay

The tissues are homogenized using a “FastPrep Cell Disrupter FP120” apparatus (Bio 101/Savant). The samples are then centrifuged at 1 000 revolutions/minute for 5 minutes. The quantity of CAT transgene expressed is determined using a standard CAT ELISA procedure (Roche, Ind.).

5) Results

The dose-response curve for compound D on the gene transfer activity of DNA/cationic lipid/DOPE complexes was studied. The compound “Analog D” was used as negative control. As expected, the highest gene transfer occurs in the lungs. It was observed that the use of the compound “Analog D” inhibits the transfection activity in the lungs and the tumors in a dose-dependent manner. By increasing the quantity of compound D, the transfection activity is also inhibited in the lungs. On the other hand, gene transfer does not appear to be affected in the tumors. These results are consistent with the hypothesis that the acidic environment of the tumors leads to a separation of the PEG parts from the rest of the particle after extravasation.

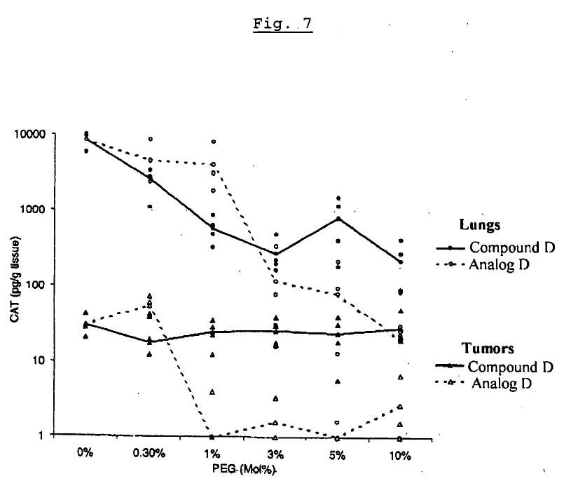

The results obtained and presented in FIG. 7 thus demonstrate that compound D makes it possible to increase the gene transfer activity specific to the tumors relative to the lungs by a factor of 40.

### Example 12

### Study of the Stability of the Acid-Sensitive Compounds as a Function of the pH

This example illustrates the fact that the acid-sensitive compounds according to the present invention have an acid-sensitivity which is modulable according to the nature of the ortho-ester ring present (5- or 6-membered ring).

To this effect, the variation of the size of nucleolipid complexes (identical to those used in examples 9 and 10) is measured as a function of the pH and time, for various acid-sensitive pegoylated lipids, which makes it possible to cover different ranges of sensitivity. These studies are carried out at fixed control cationic lipid/DNA and acid-sensitive pegoylated lipid/DNA ratios.

1 ml samples of DNA/control cationic lipid/acid-sensitive pegoylated lipid complex are prepared such that:
the control cationic lipid/DNA ratio is 1.5 nmol/μg,
the acid-sensitive pegoylated lipid/DNA ratio is 0.5 or 1, and
the DNA concentration is 20 μg/ml in 75 mM of a sodium chloride solution.

After 30 minutes, 500 μl of a buffer solution at 0.05 mol/l and 500 μl of a sodium chloride solution at 150 mM are added to these samples thermostated at 37° C. in a ventilated oven. An acidic pH is thus established and the size of the particles is measured as a function of time.

The final concentration of the samples is 10 μg of DNA/ml in 75 mM of a sodium chloride solution. The buffers used are citric acid/sodium citrate buffers at pH 4, pH 5 and pH 6 and a Hepes/sodium hydroxide buffer at pH 7.4.

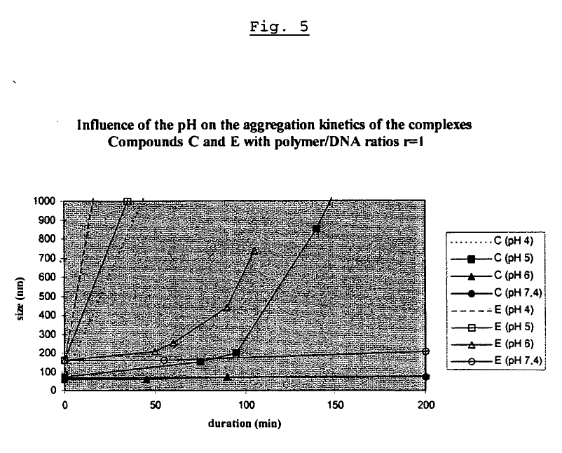

The results obtained are represented in FIG. 5 which represents the variation in the size of the nucleolipid particles as a function of the pH and of time for the acid-sensitive compounds C and E prepared in the preceding examples, and which differ only in the nature of the ortho-ester ring used (5- or 6-membered ring).

For these two compounds, an increase is observed in the size of the nucleolipid particles as a function of time when the pH is acidic, which reflects their degradation. On the other hand, an increase in the size of the nucleolipid particles as a function of time is not observed when the pH is 7.4. In addition, the lower the pH, the more rapid the aggregation of the nucleolipid particles.

Finally, a large difference in kinetics is observed between the two compounds C and E tested: at pH 6, the aggregation of the nucleolipid particles starts about 1 hour after the acidification in the case of the use of compound E, whereas the use of the compound C allows stabilization for at least 4 hours.

These results demonstrate several remarkable properties of the acid-sensitive compounds C and E, and more generally of the acid-sensitive compounds of this type according to the present application:
they both exhibit high sensitivity at acidic pH values, and in all cases, it is observed that the lower the pH, the more rapid their destabilization,
they are both relatively stable at physiological pH (pH of 7.4), and
their kinetics of degradation is very different depending on the ortho-ester group used (5- or 6-membered ring).

1. Acid-sensitive compounds characterized in that they comprise a cyclic ortho-ester and at least one hydrophilic substituent chosen from polyalkylene glycols, mono- or polysaccharides, hydrophilic therapeutic molecules, or alternatively radicals of the polyamine type, as well as their salts.

2. Acid-sensitive compounds according to claim 1, characterized in that they have the general formula: 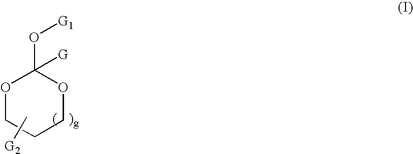
in which:
g is an integer which may take the values 0, 1, 2, 3 or 4,
G represents a hydrogen atom, an alkyl radical containing 1 to 6 carbon atoms in the form of a saturated or unsaturated, straight or branched chain, or an aryl radical,
G₁ and G₂ represent:
(a) one a hydrophilic substituent chosen from radicals of the polyamine type, and the other a hydrophobic substituent chosen from single- or double-chain alkyls, steroid derivatives or hydrophobic dendrimers, or alternatively
(b) one a hydrophobic linear alkyl group comprising 10 to 24 carbon atoms and optionally comprising one or more unsaturations, and the other a group of general formula: 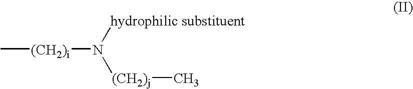
in which i is an integer ranging from 1 to 4 and j is an integer ranging from 9 to 23, and the hydrophilic substituent is chosen from radicals of the polyamine type, or alternatively
(c) one a hydrophilic substituent chosen from polyalkylene glycols or mono- or polysaccharides and the other a substituent chosen from polyalkylene imines, or alternatively
(d) one a hydrophilic substituent chosen from polyalkylene glycols or mono- or polysaccharides and the other a hydrophobic substituent chosen from single- or double-chain alkyls, steroid derivatives, hydrophobic dendrimers, or the covalent conjugates between a single- or double-chain alkyl, a steroid derivative, or a hydrophobic dendrimer and a polyalkylene glycol molecule comprising 1 to 20 monomeric units, or alternatively
(e) one a hydrophilic substituent chosen from polyalkylene glycols or mono- or polysaccharides and the other a therapeutic molecule, or alternatively
(f) one a therapeutic molecule of a hydrophilic nature and the other a hydrophobic substituent chosen from single- or double-chain alkyls, steroid derivatives or hydrophobic dendrimers,
as well as their salts.

3. Acid-sensitive compounds according to claim 2, characterized in that G is chosen from hydrogen, methyl, ethyl or phenyl.

4. Acid-sensitive compounds according to claim 2, characterized in that the single- or double-chain alkyls consist of one or two linear alkyl chains comprising 10 to 24 carbon atoms and optionally comprising one or more unsaturations.

5. Acid-sensitive compounds according to claim 2, characterized in that the steroid derivative is chosen from sterols, steroids and steroid hormones.

6. Acid-sensitive compounds according to claim 2, characterized in that the hydrophobic dendrimer is poly(benzyl ether).

7. Acid-sensitive compounds according to claim 2, characterized in that the polyalkylene glycols are chosen from polyalkylene glycols having an average molecular weight of between 10² and 10⁵ Daltons.

8. Acid-sensitive compounds according to claim 7, characterized in that the polyalkylene glycols are chosen from polyethylene glycols (PEG) having an average molecular weight of between 10² and 10⁵ Daltons.

9. Acid-sensitive compounds according to claim 2, characterized in that the mono- or polysaccharides are chosen from pyranoses, furanoses, dextrans, α-amylose, amylopectin, fructans, mannans, xylans and arabinans.

10. Acid-sensitive compounds according to claim 2, characterized in that the polyalkylene glycol or the mono- or polysaccharide is covalently linked to a targeting element.

11. Acid-sensitive compounds according to claim 10, characterized in that the targeting element is chosen from sugars, peptides, proteins, oligonucleotides, lipids, neuromediators, hormones, vitamins or their derivatives.

12. Acid-sensitive compounds according to claim 2, characterized in that the polyalkyleneimines are chosen from the polymers comprising the monomeric units of general formula: 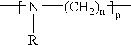
in which R may be a hydrogen atom or a group of formula: 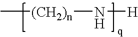
and n is an integer of between 2 and 10, p and q are integers chosen such that the sum p+q is such that the average molecular weight of the polymer is between 100 and 10⁷ Da, it being understood that the value of n may vary between the different units —NR—(CH₂)n-.

13. Acid-sensitive compounds according to claim 2, characterized in that each of the substituents G₁ and G₂ is indirectly linked to the cyclic ortho-ester via a “spacer” molecule.

14. Acid-sensitive compounds according to claim 13, characterized in that said “spacer” molecule is chosen from alkyls (1 to 6 carbon atoms), carbonyl, ester, ether, amide, carbamate or thiocarbamate bonds, glycerol, urea, thiourea or a combination of several of these groups.

15. Acid-sensitive compounds according to claim 2, characterized in that the therapeutic molecules are chosen from peptides, oligopeptides, proteins, antigens and their antibodies, enzymes and their inhibitors, hormones, antibiotics, analgesics, bronchodilators, antimicrobials, antihypertensive agents, cardiovascular agents, agents acting on the central nervous system, antihistamines, antidepressants, tranquilizers, anticonvulsants, anti-inflammatory substances, stimulants, antiemetics, diuretics, antispasmodics, antiischemics, agents limiting cell death, or anticancer agents.

16. Compositions characterized in that they comprise at least one acid-sensitive compound as defined in claims 1 to 15.

17. Compositions characterized in that they comprise at least one biologically active substance and an acid-sensitive compound as defined in claim 2 and for which G₁ and G₂ have the definitions indicated under (a), (b), (c) or (d).

18. Compositions according to claim 17, characterized in that said biologically active substance is either a therapeutic molecule as defined in claim 15, or a nucleic acid.

19. Compositions according to either of claims 16 and 17, characterized in that they comprise, in addition, one or more adjuvants.

20. Compositions according to claim 19, characterized in that said adjuvant is one or more neutral lipids.

21. Compositions according to claim 20, characterized in that said adjuvant is chosen from natural or synthetic, zwitterionic lipids or lipids lacking an ionic charge under physiological conditions.

22. Compositions according to claim 21, characterized in that said adjuvant is chosen from dioleoylphosphatidylethanolamine (DOPE), oleoyl-palmitoylphosphatidylethanolamine (POPE), distearoyl-phosphatidylethanolamine, dipalmitoylphosphatidyl-ethanolamine, dimirystoylphosphatidylethanolamine as well as their derivative which are N-methylated 1 to 3 times, phosphatidylglycerols, diacylglycerols, glycosyldiacylglycerols, cerebrosides (such as in particular galactocerebrosides), sphingolipids (such as in particular sphingomyelins) or alternatively asialogangliosides (such as in particular asialoGM1 and GM2).

23. Compositions according to one of claims 16 to 22, characterized in that they comprise, in addition, a pharmaceutically acceptable vehicle for an injectable formulation.

24. Compositions according to one of claims 16 to 22, characterized in that they comprise, in addition, a pharmaceutically acceptable vehicle for administration to the skin and/or the mucous membranes.

25. Use of an acid-sensitive compound as defined in claims 1 to 15 for the manufacture of a medicament intended for treating diseases.

26. Use of an acid-sensitive compound as defined as in claim 2 and for which G₁ and G₂ have the definitions indicated under (a), (b), (c) or (d), for the manufacture of a medicament intended for the transfection of nucleic acids.

27. Acid-sensitive compounds as defined in claim 2 and for which G₁ and G₂ have the definitions indicated under (e) or (f) for use as a medicament.
